(19) United States
(12) Patent Application Publication
(10) Pub. No.: US 2013/0165696 A1
(43) Pub. Date: Jun. 27, 2013

(54) NOVEL PROCESSES FOR THE PREPARATION OF PHENYLCYCLOPROPYLAMINE DERIVATIVES AND USE THEREOF FOR PREPARING TICAGRELOR

(71) Applicants: Anil Shahaji Khile, Raigad (IN); Vignesh Nair, Kerala (IN); Nikhil Trivedi, Maharashtra (IN); Nitin Sharadchandra Pradhan, Maharashtra (IN)

(72) Inventors: Anil Shahaji Khile, Raigad (IN); Vignesh Nair, Kerala (IN); Nikhil Trivedi, Maharashtra (IN); Nitin Sharadchandra Pradhan, Maharashtra (IN)

(73) Assignee: ACTAVIS GROUP PTC EHF, Hafnarfjordur (IS)

(21) Appl. No.: 13/805,150

(22) PCT Filed: Jun. 28, 2011
(86) PCT No.: PCT/IB2011/002246

§ 371 Date: Mar. 11, 2013

(30) Foreign Application Priority Data
Jun. 30, 2010 (IN) 1841/CHE/2010
Jul. 19, 2010 (IN) 2043/CHE/2010

(51) Int. Cl. C07C 209/46

(52) U.S. Cl. CPC C07C 209/46
USPC 564/307

(57) ABSTRACT

Provided herein are novel processes for the preparation of phenylcyclopropylamine derivatives, which are useful intermediates in the preparation of triazolo[4,5-d]pyrimidine compounds. Provided particularly herein are novel, commercially viable and industrially advantageous processes for the preparation of a substantially pure ticagrelor intermediate, trans-(1R,2S)-2-(3,4-difluorophenyl)-cyclopropylamine. Provided further herein are novel acid addition salts of trans-(1R,2S)-2-(3,4-difluorophenyl)-cyclopropylamine, and process for their preparation. The intermediate and its acid addition salts are useful for preparing ticagrelor, or a pharmaceutically acceptable salt thereof, in high yield and purity.

### CROSS REFERENCE TO RELATED APPLICATIONS

This application claims the benefit of priority to Indian provisional application Nos. 1841/CHE/2010, filed on Jun. 30, 2010; and 2043/CHE/2010, filed on Jul. 19, 2010; which are incorporated herein by reference in their entirety.

### FIELD OF THE DISCLOSURE

The present disclosure relates to novel processes for the preparation of phenylcyclopropylamine derivatives, which are useful intermediates in the preparation of triazolo[4,5-d]pyrimidine compounds. The present disclosure particularly relates to novel, commercially viable and industrially advantageous processes for the preparation of a substantially pure ticagrelor intermediate, trans-(1R,2S)-2-(3,4-difluorophenyl)-cyclopropylamine. The present disclosure further relates to novel acid addition salts of trans-(1R,2S)-2-(3,4-difluorophenyl)-cyclopropylamine, and process for their preparation. The intermediate and its acid addition salts are useful for preparing ticagrelor, or a pharmaceutically acceptable salt thereof, in high yield and purity.

### BACKGROUND

U.S. Pat. Nos. 6,251,910 and 6,525,060 disclose a variety of triazolo[4,5-d]pyrimidine derivatives, processes for their preparation, pharmaceutical compositions comprising the derivatives, and methods of use thereof. These compounds act as P_{2T} (P2Y_{ADP} or P2T_{AC}) receptor antagonists and they are indicated for use in therapy as inhibitors of platelet activation, aggregation and degranulation, promoters of platelet disaggregation, and anti-thrombotic agents. Among them, Ticagrelor, [1S-(1α,2α,3β(1S*,2R*),5β)]-3-[7-[2-(3,4-difluorophenyl)cyclo propyl]amino]-5-(propylthio)-3H-1,2,3-triazolo[4,5-d]pyrimidin-3-yl)-5-(2-hydroxyethoxy)-cyclopentane-1,2-diol, acts as an adenosine uptake inhibitor, a platelet aggregation inhibitor, a P2Y12 purinoceptor antagonist, and a coagulation inhibitor. It is indicated for the treatment of thrombosis, angina, ischemic heart diseases, and coronary artery diseases. Ticagrelor is represented by the following structural formula I:

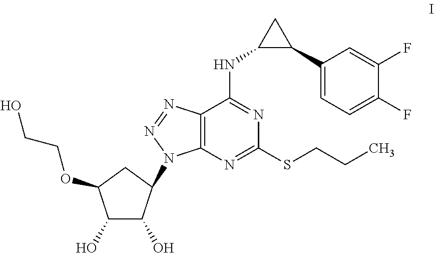 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is the first reversibly binding oral adenosine diphosphate (ADP) receptor antagonist and is chemically distinct from thienopyridine compounds like clopidogrel. It selectively inhibits P2Y12, a key target receptor for ADP. ADP receptor blockade inhibits the action of platelets in the blood, reducing recurrent thrombotic events. The drug has shown a statistically significant primary efficacy against the widely prescribed clopidogrel (Plavix) in the prevention of cardiovascular (CV) events including myocardial infarction (heart attacks), stroke, and cardiovascular death in patients with acute coronary syndrome (ACS).

Various processes for the preparation of pharmaceutically active triazolo[4,5-d]pyrimidine cyclopentane compounds, preferably ticagrelor, their enantiomers, and their pharmaceutically acceptable salts are disclosed in U.S. Pat. Nos. 6,251,910; 6,525,060; 6,974,868; 7,067,663; 7,122,695 and 7,250,419; U.S. Patent Application Nos. 2007/0265282, 2008/0132719 and 2008/0214812; European Patent Nos. EP0996621 and EP1135391; and PCT Publication Nos. WO2008/018823 and WO2010/030224.

One of the useful intermediates in the synthesis of pharmaceutically active triazolo[4,5-d]pyrimidine cyclopentane compounds is the substituted phenylcyclopropylamine derivative of formula II:

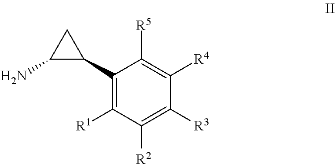

wherein R¹, R², R³, R⁴ and R⁵ are, each independently, selected from hydrogen and a halogen atom, wherein the halogen atom is F, Cl, Br or I; preferably, the halogen atom is F.

In the preparation of ticagrelor, trans-(1R,2S)-2-(3,4-difluorophenyl)-cyclopropylamine of formula IIa:

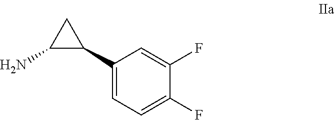 [SMILES: N[C@@H]1C[C@H]1c1ccc(F)c(F)c1]

is a key intermediate.

According to U.S. Pat. No. 6,251,910 (hereinafter referred to as the '910 patent), the substituted phenylcyclopropylamine derivatives of formula II are prepared by a process as depicted in scheme 1:

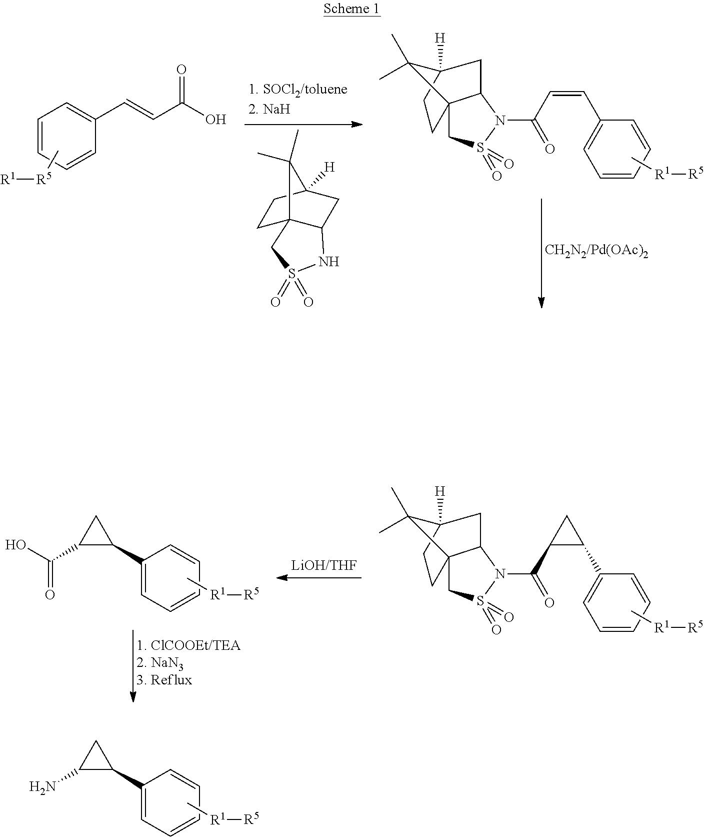

The process for the preparation of substituted phenylcyclopropylamine derivatives disclosed in the '910 patent involves the use of hazardous and explosive materials like sodium hydride, diazomethane and sodium azide. The process also involves the use of highly expensive chiral sultam auxiliary. Moreover, the yields of substituted phenylcyclopropylamine derivatives obtained are low to moderate, and the process involves column chromatographic purifications.

Methods involving column chromatographic purifications are generally undesirable for large-scale operations, thereby making the process commercially unfeasible. The use of explosive reagents like sodium hydride, diazomethane and sodium azide is not advisable, due to the handling difficulties, for scale up operations.

U.S. Pat. No. 7,122,695 (hereinafter referred to as the '695 patent) discloses a process for the preparation of substituted phenylcyclopropylamine derivatives, specifically trans-(1R,2S)-2-(3,4-difluorophenyl)cyclopropylamine and its mandelate salt. The synthesis is depicted in scheme 2:

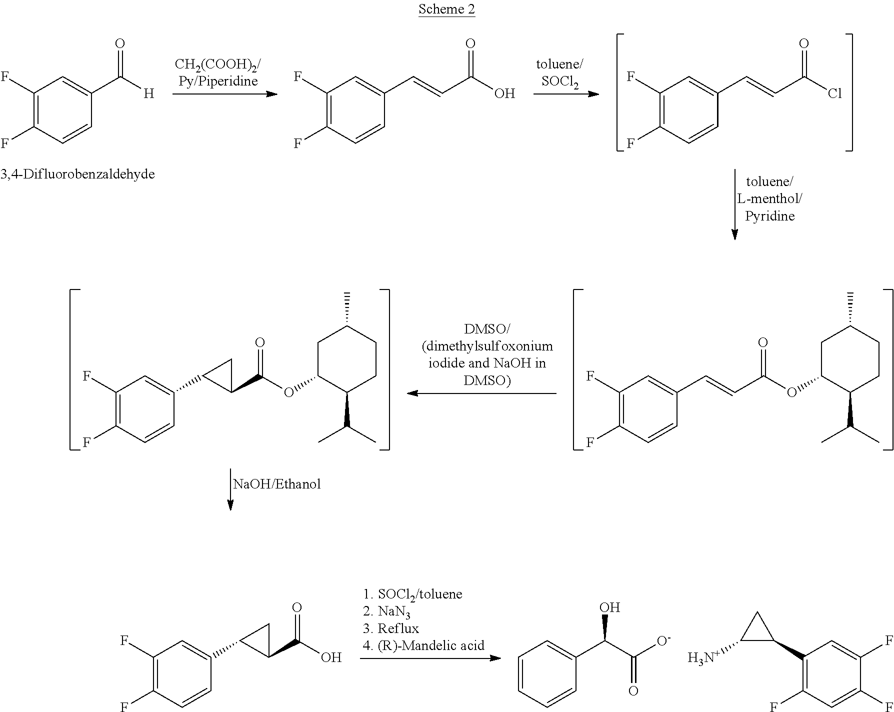

According to the '695 patent, the trans-(1R,2S)-2-(3,4-difluorophenyl)cyclopropylamine is prepared by reacting 3,4-difluorobenzaldehyde with malonic acid in the presence of pyridine and piperidine to produce (E)-3-(3,4-difluorophenyl)-2-propenoic acid, followed by the reaction with thionyl chloride in the presence of pyridine in toluene to produce (E)-3-(3,4-difluorophenyl)-2-propenoyl chloride, which is then reacted with L-menthol in the presence of pyridine in toluene to produce (1R,2S,5R)-2-isopropyl-5-methylcyclohexyl (E)-3-(3,4-difluorophenyl)-2-propenoate. The (1R,2S,5R)-2-isopropyl-5-methylcyclohexyl (E)-3-(3,4-difluorophenyl)-2-propenoate is then reacted with dimethylsulfoxonium methylide in the presence of sodium hydroxide and sodium iodide in dimethylsulfoxide to produce a solution containing (1R,2S,5R)-2-isopropyl-5-methylcyclohexyl trans-2-(3,4-difluorophenyl)cyclopropanecarboxylate, followed by the diastereomeric separation to produce (1R,2S,5R)-2-isopropyl-5-methylcyclohexyl trans-(1R,2R)-2-(3,4-difluorophenyl)cyclopropanecarboxylate. The ester compound is hydrolyzed with sodium hydroxide in ethanol, followed by the acidification with hydrochloric acid to produce trans-(1R,2R)-2-(3,4-difluorophenyl)cyclopropanecarboxylic acid, followed by reaction with thionyl chloride in the presence of pyridine in toluene to produce trans-(1R,2R)-2-(3,4-difluorophenyl)cyclopropanecarbonyl chloride, which is then reacted with sodium azide in the presence of tetrabutylammonium bromide and sodium carbonate in toluene to produce a reaction mass containing trans-(1R,2R)-2-(3,4-difluorophenyl)cyclopropanecarbonyl azide. The azide compound is then added to toluene while stirring at 100° C., followed by acid/base treatment to produce trans-(1R,2R)-2-(3,4-difluorophenyl)cyclopropylamine, which is then converted to its mandelate salt by reaction with R-(−)-mandelic acid in ethyl acetate.

The process disclosed in the '695 patent is lengthy thus resulting in a poor product yield. The process also involves the use of hazardous materials like pyridine and sodium azide.

U.S. Patent Application No. 2008/0132719 (hereinafter referred to as the '719 application) describes a process for the preparation of (1R,2S)-2-(3,4-difluorophenyl)-cyclopropane amine. The synthetic route is depicted in scheme 3:

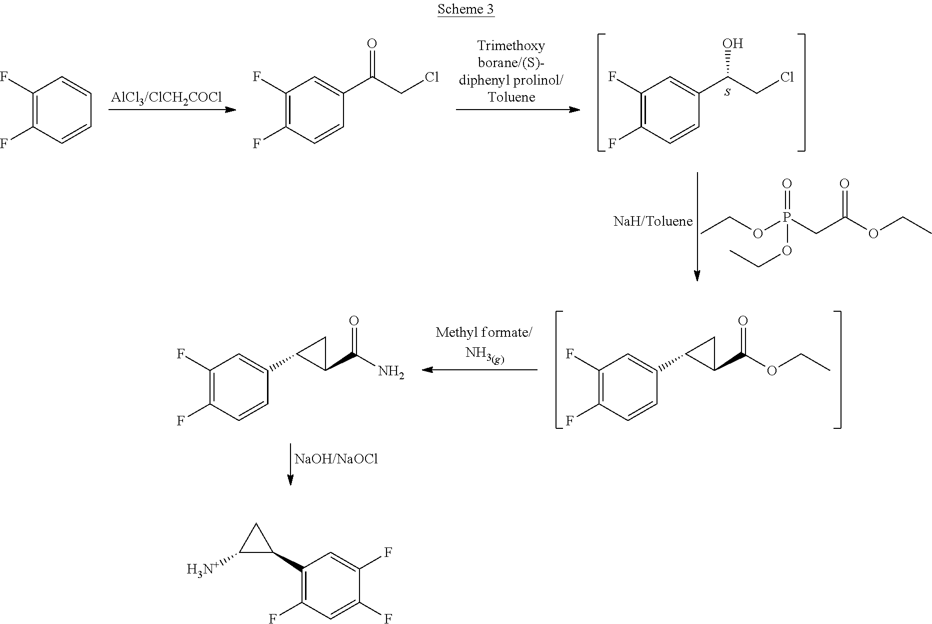

According to the '719 application, the (1R,2S)-2-(3,4-difluorophenyl)-cyclopropane amine is prepared by reacting 1,2-difluorobenzene with chloroacetyl chloride in the presence of aluminium trichloride to produce 2-chloro-1-(3,4-difluorophenyl)ethanone, followed by the reaction with trimethoxy borane and S-diphenylprolinol in toluene to produce 2-chloro-(1S)-(3,4-difluorophenyl)ethanol, which is then reacted with triethyl phosphonoacetate in the presence of sodium hydride in toluene to produce ethyl (1R,2R)-trans-2-(3,4-difluorophenyl)cyclopropyl carboxylate. The ester compound is then reacted with methyl formate in the presence of ammonia to produce (1R,2R)-trans-2-(3,4-difluorophenyl)cyclopropyl carboxamide, which is then reacted with sodium hydroxide and sodium hypochlorite to produce (1R,2S)-2-(3,4-difluorophenyl)-cyclopropane amine.

The process described in the '719 application suffers from the disadvantages such as the use of explosive materials like sodium hydride.

PCT Publication No. WO2008/018823 (hereinafter referred to as the '823 publication) describes a process for the preparation of (1R,2S)-2-(3,4-difluorophenyl)-1-cyclopropanamine. The synthetic route is depicted in scheme 4:

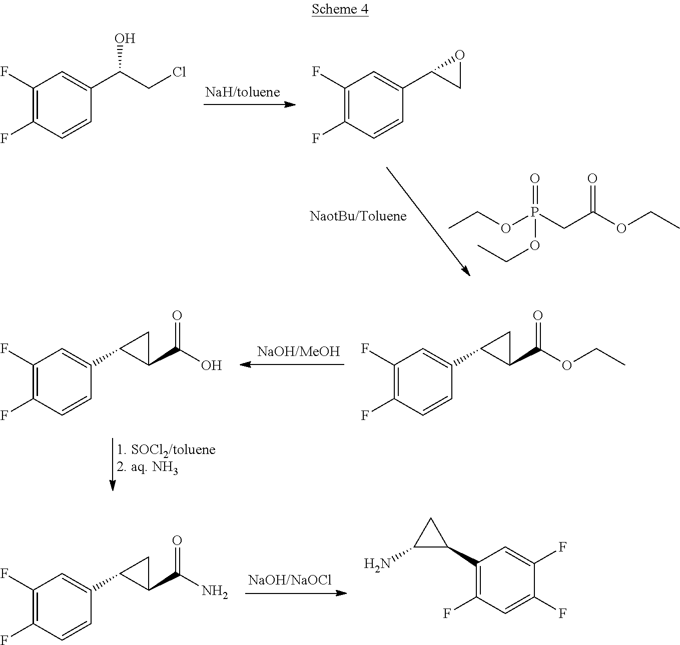

According to the '823 publication, the (1R,2S)-2-(3,4-difluorophenyl)-1-cyclopropanamine is prepared by reacting (1S)-2-chloro-1-(3,4-difluorophenyl)-1-ethanol with sodium hydroxide in toluene to produce (2S)-2-(3,4-difluorophenyl)oxirane, followed by reaction with triethyl phosphonoacetate in the presence of sodium t-butoxide in toluene to produce ethyl (1R,2R)-2-(3,4-difluorophenyl)-1-cyclopropanecarboxylate, which is then hydrolyzed with sodium hydroxide in methanol to produce (1R,2R)-2-(3,4-difluorophenyl)-1-cyclopropanecarboxylic acid. The resulting carboxylic acid compound is reacted with thionyl chloride in toluene to produce a solution of (1R,2R)-2-(3,4-difluorophenyl)-1-cyclopropanecarbonyl chloride, followed by subsequent reaction with aqueous ammonia to produce (1R,2R)-2-(3,4-difluorophenyl)-1-cyclopropanecarboxamide, which is then reacted with sodium hydroxide in the presence of sodium hypochlorite to produce (1R,2S)-2-(3,4-difluorophenyl)-1-cyclopropanamine.

Bioorganic & Medicinal Chemistry, vol. 17(6), pages 2388-2399 (2009) discloses a process for the preparation of racemic trans-2-(3,4-difluorophenyl)cyclopropylamine and its acid addition salt.

J. Med. Chem., vol. 20, No. 7, pages 934-939 (1977) discloses a process for the preparation of 1-aryl-3-nitro-1-propanones from 1-aryl-3-chloro-1-propanones.

J. Org. Chem. 57, pages 3757-3759 (1992) discloses an intramolecular Mitsunobu displacement with carbon nucleophiles for preparation of nitrocyclopropanes from nitroalkanol.

Based on the aforementioned drawbacks, the prior art processes have been found to be unsuitable for the preparation of substituted phenylcyclopropylamine derivatives of formula II at lab scale and in commercial scale operations.

A need remains for an improved and commercially viable process of preparing substituted phenylcyclopropylamine derivatives of formula II with high yields and purity, to resolve the problems associated with the processes described in the prior art, and that will be suitable for large-scale preparation. Furthermore, there remains a need for novel acid addition salts of trans-(1R,2S)-2-(3,4-difluorophenyl)-cyclopropylamine and use thereof for preparing highly pure ticagrelor or a pharmaceutically acceptable salt thereof. Desirable process properties include non-hazardous conditions, environmentally friendly and easy to handle reagents, reduced reaction times, reduced cost, greater simplicity, increased purity, and increased yield of the product, thereby enabling the production of triazolo[4,5-d]pyrimidinecyclopentane compounds, preferably ticagrelor, and their pharmaceutically acceptable acid addition salts in high purity and with high yield.

### SUMMARY

In one aspect, provided herein are novel, efficient, industrially advantageous and environmentally friendly processes for the preparation of substituted phenylcyclopropylamine derivatives using novel intermediates, preferably trans-(1R,2S)-2-(3,4-difluorophenyl)-cyclopropylamine or an acid addition salt thereof, in high yield, and with high chemical and enantiomeric purity. Moreover, the processes disclosed herein involve non-hazardous and easy to handle reagents, reduced reaction times, and reduced synthesis steps. The processes avoid the tedious and cumbersome procedures of the prior processes and are convenient to operate on a commercial scale.

In another aspect, the present disclosure also encompasses the use of pure trans-(1R,2S)-2-(3,4-difluorophenyl)-cyclopropylamine or an acid addition salt thereof obtained by the processes disclosed herein for preparing ticagrelor or a pharmaceutically acceptable salt thereof.

In another aspect, provided herein are novel acid addition salts of trans-(1R,2S)-2-(3,4-difluorophenyl)-cyclopropylamine, wherein the acid addition salt is a tartrate salt, a di-p-toluoyl-tartrate salt, an (S)-ketopinate salt, a (D)-malate salt, a (D)-camphorsulfonate salt, a (R)-(−)-α-methoxyphenyl acetate salt, a fumarate salt, a phosphate salt, or a sulfate salt.

In another aspect, the acid addition salts of trans-(1R,2S)-2-(3,4-difluorophenyl)-cyclopropylamine in a solid state form are provided. In another aspect, the acid addition salts of trans-(1R,2S)-2-(3,4-difluorophenyl)-cyclopropylamine in a crystalline form are provided. In yet another aspect, the acid addition salts of trans-(1R,2S)-2-(3,4-difluorophenyl)-cyclopropylamine in an amorphous form are provided.

The process for the preparation of substituted phenylcyclopropylamine derivatives disclosed herein has the following advantages over the processes described in the prior art:
i) the overall process involves a reduced number of process steps and shorter reaction times;
ii) the process avoids the use of hazardous or explosive chemicals like sodium hydride, diazomethane, pyridine and sodium azide;
iii) the process avoids the use of tedious and cumbersome procedures like column chromatographic purifications and multiple isolations;
iv) the process avoids the use of expensive materials like chiral sultam auxiliary;
v) the process involves easy work-up methods and simple isolation processes, and there is a reduction in chemical waste;
vi) the purity of the product is increased without additional purifications; and
vii) the overall yield of the product is increased.

### BRIEF DESCRIPTION OF THE DRAWINGS

FIG. 1 is a characteristic powder X-ray diffraction (XRD) pattern of crystalline trans-(1R,2S)-2-(3,4-difluorophenyl)-cyclopropylamine tartrate salt.

FIG. 2 is a characteristic differential scanning calorimetric (DSC) thermogram of crystalline trans-(1R,2S)-2-(3,4-difluorophenyl)-cyclopropylamine tartrate salt.

FIG. 3 is a characteristic powder X-ray diffraction (XRD) pattern of crystalline trans-(1R,2S)-2-(3,4-difluorophenyl)-cyclopropylamine di-p-toluoyl-tartrate salt.

FIG. 4 is a characteristic powder X-ray diffraction (XRD) pattern of crystalline trans-(1R,2S)-2-(3,4-difluorophenyl)-cyclopropylamine (S)-ketopinate salt.

FIG. 5 is a characteristic differential scanning calorimetric (DSC) thermogram of crystalline trans-(1R,2S)-2-(3,4-difluorophenyl)-cyclopropylamine (S)-ketopinate salt.

FIG. 6 is a characteristic powder X-ray diffraction (XRD) pattern of crystalline trans-(1R,2S)-2-(3,4-difluorophenyl)-cyclopropylamine (D)-malate salt.

FIG. 7 is a characteristic differential scanning calorimetric (DSC) thermogram of crystalline trans-(1R,2S)-2-(3,4-difluorophenyl)-cyclopropylamine (D)-malate salt.

FIG. 8 is a characteristic powder X-ray diffraction (XRD) pattern of crystalline trans-(1R,2S)-2-(3,4-difluorophenyl)-cyclopropylamine (D)-camphorsulfonate salt.

FIG. 9 is a characteristic differential scanning calorimetric (DSC) thermogram of crystalline trans-(1R,2S)-2-(3,4-difluorophenyl)-cyclopropylamine (D)-camphorsulfonate salt.

FIG. 10 is a characteristic powder X-ray diffraction (XRD) pattern of crystalline trans-(1R,2S)-2-(3,4-difluorophenyl)-cyclopropylamine (R)-(−)-α-methoxyphenylacetate salt.

FIG. 11 is a characteristic powder X-ray diffraction (XRD) pattern of crystalline trans-(1R,2S)-2-(3,4-difluorophenyl)-cyclopropylamine fumarate salt.

FIG. 12 is a characteristic differential scanning calorimetric (DSC) thermogram of crystalline trans-(1R,2S)-2-(3,4-difluorophenyl)-cyclopropylamine fumarate salt.

FIG. 13 is a characteristic powder X-ray diffraction (XRD) pattern of crystalline trans-(1R,2S)-2-(3,4-difluorophenyl)-cyclopropylamine phosphate salt.

FIG. 14 is a characteristic differential scanning calorimetric (DSC) thermogram of crystalline trans-(1R,2S)-2-(3,4-difluorophenyl)-cyclopropylamine phosphate salt.

FIG. 15 is a characteristic powder X-ray diffraction (XRD) pattern of crystalline trans-(1R,2S)-2-(3,4-difluorophenyl)-cyclopropylamine sulfate salt.

FIG. 16 is a characteristic differential scanning calorimetric (DSC) thermogram of crystalline trans-(1R,2S)-2-(3,4-difluorophenyl)-cyclopropylamine sulfate salt.

### DETAILED DESCRIPTION

According to one aspect, there is provided a process for preparing substituted phenylcyclopropylamine derivatives of formula II:

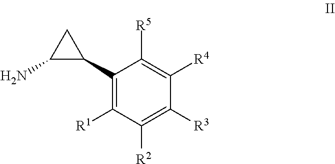

or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I, preferably, the halogen atom is F; comprising:
a) reacting a halogen substituted benzaldehyde compound of formula VIII:

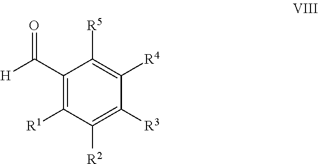

wherein R¹, R², R³, R⁴ and R⁵ are as defined in formula II; with a methyltriphenyl phosphonium halide (Wittig reagent) of formula VII:

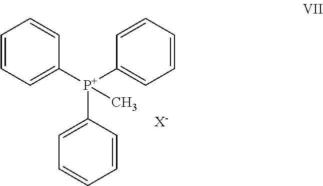

wherein ‘X’ is a halogen, selected from the group consisting of Cl, Br and I;
in the presence of a first base in a first solvent to produce a substituted styrene compound of formula VI:

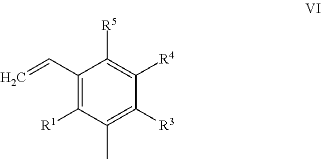

wherein R¹, R², R³, R⁴ and R⁵ are as defined above;
b) reacting the compound of formula VI with a diazoester compound of formula V:

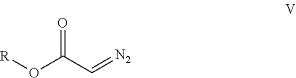

wherein ‘R’ is an alkyl, cycloalkyl, aryl or aralkyl group; in the presence of a metal catalyst and a chiral ligand in a second solvent to produce a substituted cyclopropanecarboxylate compound of formula IV:

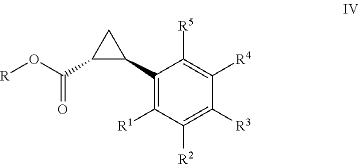

or a stereochemically isomeric form or a mixture of stereochemically isomeric forms thereof, wherein R, R¹, R², R³, R⁴ and R⁵ are as defined above;
c) hydrolyzing the ester compound of formula IV with an acid or a second base in a third solvent to produce a substituted cyclopropanecarboxylic acid compound of formula III:

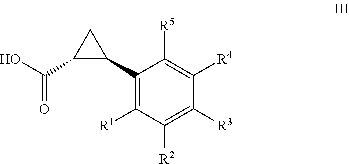

or a stereochemically isomeric form or a mixture of stereochemically isomeric forms thereof;
d) optionally, purifying the cyclopropanecarboxylic acid compound of formula III by treating with a chiral amine in a fourth solvent to produce a pure chiral amine salt of the compound of formula III;
e) optionally, acidifying the chiral amine salt of the compound of formula III with an acid to produce a pure cyclopropanecarboxylic acid compound of formula III;
f) reacting the cyclopropanecarboxylic acid compound of formula III or a chiral amine salt thereof obtained in step-(c), (d) or (e) with an azide compound, with the proviso that the azide does not include sodium azide, in the presence a third base in a fifth solvent to produce an isocyanate intermediate, followed by subjecting to acidic hydrolysis with an acid in a sixth solvent and then basifying with a fourth base to produce the substituted phenylcyclopropylamine derivatives of formula II or a stereochemically isomeric form or a mixture of stereochemically isomeric forms thereof, and optionally converting the compound of formula II obtained into an acid addition salt thereof.

In one embodiment, the halogen atom ‘X’ in the compound of formula VII is Cl or Br, and more specifically, X is Br.

In another embodiment, in the compounds of formulae II, III, IV, VI and VIII, the R¹, R² and R⁵ are H, and wherein the R³ and R⁴ are F.

The compounds of formulae II, III and IV can exist in different isomeric forms such as cis/trans isomers, enantiomers, or diastereomers. The process disclosed herein includes all such isomeric forms and mixtures thereof in all proportions.

The term “alkyl”, as used herein, denotes an aliphatic hydrocarbon group which may be straight or branched having 1 to 12 carbon atoms in the chain. Preferred alkyl groups have 1 to 6 carbon atoms in the chain. The alkyl may be substituted with one or more “cycloalkyl group”. Exemplary alkyl groups include methyl, ethyl, n-propyl, iso-propyl, n-butyl, iso-butyl, t-butyl, n-pentyl, cyclopentylmethyl.

The term “cycloalkyl”, as used herein, denotes a non-aromatic mono- or multicyclic ring system of 3 to 10 carbon atoms, preferably of about 5 to about 10 carbon atoms. Exemplary monocyclic cycloalkyl groups include cyclopentyl, cyclohexyl, cycloheptyl and the like.

The term “aralkyl”, as used herein, denotes an aryl-alkyl group wherein the aryl and alkyl are as herein described. Preferred aralkyls contain a lower alkyl moiety. Exemplary aralkyl groups include benzyl, 2-phenethyl and naphthalenemethyl.

The term “aryl”, as used herein, denotes an aromatic monocyclic or multicyclic ring system of 6 to 10 carbon atoms. The aryl is optionally substituted with one or more “ring system substituents” which may be the same or different, and are as defined herein. Exemplary aryl groups include phenyl or naphthyl.

Specifically, the group ‘R’ in the compounds of formulae IV and V is selected from the group consisting of methyl, ethyl, isopropyl, tert-butyl, benzyl, 1- or d-menthyl, and the like; and more specifically, R is ethyl.

In one embodiment, a specific substituted phenylcyclopropylamine derivative of formula II prepared by the processes described herein is trans-(1R,2S)-2-(3,4-difluorophenyl)-cyclopropylamine of formula IIa (formula II, wherein R¹, R² and R⁵ are H, and R³ and R⁴ are F):

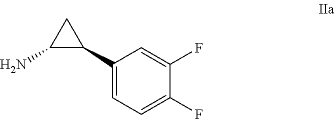 [SMILES: N[C@@H]1C[C@H]1c1ccc(F)c(F)c1]

In another embodiment, a specific substituted phenylcyclopropylamine derivative of formula II prepared by the processes described herein is trans-(1S,2R)-2-(3,4-difluorophenyl)-cyclopropylamine of formula IIb (formula II, wherein R¹, R² and R⁵ are H, and R³ and R⁴ are F):

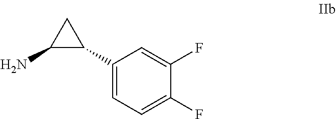 [SMILES: N[C@H]1C[C@@H]1c1ccc(F)c(F)c1]

Exemplary first solvents used in step-(a) include, but are not limited to, an ester, a nitrile, a hydrocarbon, a cyclic ether, an aliphatic ether, a polar aprotic solvent, and mixtures thereof. The term solvent also includes mixtures of solvents.

Specifically, the first solvent is selected from the group consisting of ethyl acetate, isopropyl acetate, isobutyl acetate, tert-butyl acetate, acetonitrile, propionitrile, tetrahydrofuran, 2-methyl-tetrahydrofuran, 1,4-dioxane, methyl tert-butyl ether, diethyl ether, diisopropyl ether, monoglyme, diglyme, n-hexane, n-heptane, cyclohexane, toluene, xylene, N,N-dimethylformamide, N,N-dimethylacetamide, dimethylsulfoxide, N-methylpyrrolidone, and mixtures thereof; and a most specific solvent is toluene.

In one embodiment, the first base used in step-(a) is an organic or inorganic base. Exemplary organic bases include, but are not limited to, alkyl metals such as methyl lithium, butyl lithium, hexyl lithium; alkali metal complexes with amines such as lithium diisopropyl amide; and organic amine bases of formula NR₁R₂R₃, wherein R₁, R₂ and R₃ are independently hydrogen, C₁₋₆ straight or branched chain alkyl, aryl alkyl, or C₃₋₁₀ single or fused ring optionally substituted, alkylcycloalkyl; or independently R₁, R₂ and R₃ combine with each other to form a C₃₋₇ membered cycloalkyl ring or heterocyclic system containing one or more hetero atoms. Specific organic bases are trimethylamine, dimethyl amine, diethylamine, tert-butyl amine, tributylamine, triethylamine, diisopropylethylamine, pyridine, N-methylmorpholine, 4-(N,N-dimethylamino)pyridine, methyl lithium, butyl lithium, hexyl lithium, lithium diisopropyl amide, 1,8-diazabicyclo[5.4.0]undec-7-ene; and most specifically butyl lithium and 1,8-diazabicyclo[5.4.0]undec-7-ene.

Exemplary inorganic bases include, but are not limited to, hydroxides, alkoxides, bicarbonates and carbonates of alkali or alkaline earth metals, and ammonia. Specific inorganic bases are aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide, and more specifically sodium tert-butoxide, sodium isopropoxide and potassium tert-butoxide.

Specific Wittig reagents used in step-(a) are methyl triphenylphosphonium chloride, methyl triphenylphosphonium bromide, methyl triphenylphosphonium iodide, and more specifically methyl triphenylphosphonium bromide.

In one embodiment, the reaction in step-(a) is carried out at a temperature of about −50° C. to about 150° C. for at least 30 minutes, specifically at a temperature of about 0° C. to about 100° C. for about 2 hours to about 10 hours, and more specifically at about 35° C. to about 80° C. for about 3 hours to about 6 hours.

The reaction mass containing the substituted styrene compound of formula VI obtained in step-(a) may be subjected to usual work up such as a washing, an extraction, a pH adjustment, an evaporation or a combination thereof. The reaction mass may be used directly in the next step or the styrene compound of formula VI may be isolated and then used in the next step.

In one embodiment, the styrene compound of formula VI is isolated from a suitable solvent by conventional methods such as cooling, seeding, partial removal of the solvent from the solution, by adding an anti-solvent to the solution, evaporation, vacuum distillation, or a combination thereof.

In another embodiment, the reaction mass containing the styrene compound of formula VI obtained is concentrated and then taken for the next step.

Exemplary second solvents used in step-(b) include, but are not limited to, a ketone, an ester, a hydrocarbon, a chlorinated hydrocarbon, a cyclic ether, an aliphatic ether, and mixtures thereof. The term solvent also includes mixtures of solvents.

In one embodiment, the second solvent is selected from the group consisting of acetone, methyl ethyl ketone, methyl isobutyl ketone, methyl tert-butyl ketone, ethyl acetate, methyl acetate, isopropyl acetate, tert-butyl methyl acetate, ethyl formate, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, dichloromethane, dichloroethane, chloroform, carbon tetrachloride, and mixtures thereof; and most specifically toluene, tetrahydrofuran, 2-methyl tetrahydrofuran, and mixtures thereof.

Specific diazoester compounds of formula V used in step-(b) are ethyl diazoacetate, isopropyl diazoacetate, tert-butyl diazoacetate, benzyl diazoacetate, 1 or d-menthyl diazoacetate, butylated toluene diazoacetate, and mixtures thereof; and a most specific diazoester is ethyl diazoacetate.

Exemplary metal catalysts used in step-(b) include, but are not limited to, chlorides, bromides, acetates and fluoroalkyl acetates of metals such as cobalt, copper, chromium, iron, manganese, aluminium, ruthenium and rhodium. A most specific metal catalyst is dichloro(p-cymene)ruthenium(II) dimer.

Exemplary chiral ligands employed for facilitating the asymmetric cyclopropanation reaction in step-(b) include, but are not limited to, bisoxazoline compounds, substituted salicylaldimines, salens, optically active Schiff bases, bipyridines, bisazaferrocene, dirhodium(II)carboxylates, dirhodium(II)carboxamidates, and mixtures thereof.

Exemplary optically active bisoxazoline compounds include, but are not limited to, 2,2′-methylenebis[(4R)-4-phenyl-2-oxazoline], 2,2′-methylenebis[(4R)-4-isopropyl-2-oxazoline], 2,2′-methylenebis[(4R)-4-t-butyl-2-oxazoline], 2,2′-methylenebis[(4R)-4-benzyl-2-oxazoline], 2,2′-methylenebis[(4R,5R)-4-methyl-5-phenyl-2-oxazoline], 2,2′-methylenebis[(4R,5S)-4-benzyl-5-phenyl-2-oxazoline], 2,2′-methylenebis[(4R,5S)-4,5-diphenyl-2-oxazoline], 2,2′-methylenebis[(4R)-4-phenyl-5,5-dimethyl-2-oxazoline, 2,2′-methylenebis[(4R)-4-phenyl-5,5-diethyl-2-oxazoline], 2,2′-methylenebis[(4R)-4-phenyl-5,5-di-n-propyl-2-oxazoline], 2,2′-methylenebis[(4R)-4-phenyl-5,5-di-1-propyl-2-oxazoline], 2,2′-methylenebis[(4R)-4-phenyl-5,5-dicyclohexyl-2-oxazoline], 2,2′-methylenebis[(4R)-4-phenyl-5,5-diphenyl-2-oxazoline], 2,2′-methylenebis[(4R)-4-phenyl-5,5-di-(2-methylphenyl)-2-oxazoline], 2,2′-methylenebis[(4R)-4-phenyl-5,5-di-(3-methylphenyl)-2-oxazoline], 2,2′-methylenebis[(4R)-4-phenyl-5,5-di-(4-methylphenyl)-2-oxazoline], 2,2′-methylenebis[(4R)-4-phenyl-5,5-di-(2-methoxyphenyl)-2-oxazoline], 2,2′-methylenebis[(4R)-4-phenyl-5,5-di-(3-methoxyphenyl)-2-oxazoline], 2,2′-methylenebis[(4R)-4-phenyl-5,5-di-(4-methoxyphenyl)-2-oxazoline], 2,2′-methylenebis[spiro [(4R)-4-phenyl-2-oxazoline-5,1′-cyclobutane]], 2,2′-methylenebis[spiro[(4R)-4-phenyl-2-oxazoline-5,1′-cyclopentane]], 2,2′-methylenebis[spiro[(4R)-4-phenyl-2-oxazoline-5,1′-cyclohexane]], 2,2′-methylenebis[spiro[(4R)-4-phenyl-2-oxazoline-5,1′-cycloheptane]], 2,2′-isopropylidenebis[(4R)-4-phenyl-2-oxazoline], 2,2′-isopropylidenebis[(4R)-4-isopropyl-2-oxazoline], 2,2′-isopropylidenebis[(4R)-4-t-butyl-2-oxazoline], 2,2′-isopropylidenebis[(4R)-4-benzyl-2-oxazoline], 2,2′-isopropylidenebis[(4R,5R)-4-methyl-5-phenyl-2-oxazoline], 2,2′-isopropylidenebis[(4R,5S)-4,5-diphenyl-2-oxazoline], 2,2′-isopropylidenebis[(4R,5S)-4-benzyl-5-phenyl-2-oxazoline], 2,2′-isopropylidenebis[(4R)-4-phenyl-5,5-dimethyl-2-oxazoline], 2,2′-isopropylidenebis[(4R)-4-phenyl-5,5-diethyl-2-oxazoline], 2,2′-isopropylidenebis[(4R)-4-phenyl-5,5-di-n-propyl-2-oxazoline], 2,2′-isopropylidenebis[(4R)-4-phenyl-5,5-di-1-propyl-2-oxazoline], 2,2′-isopropylidenebis[(4R)-4-phenyl-5,5-dicyclohexyl-2-oxazoline], 2,2′-isopropylidenebis[(4R)-4-phenyl-5,5-di-phenyl-2-oxazoline], 2,2′-isopropylidenebis[(4R)-4-phenyl-5,5-di-(2-methylphenyl)-2-oxazoline], 2,2′-isopropylidenebis[(4R)-4-phenyl-5,5-di-(3-methylphenyl)-2-oxazoline], 2,2′-isopropylidenebis[(4R)-4-phenyl-5,5-di-(4-methylphenyl)-2-oxazoline], and 2,2′-isopropylidenebis[(4R)-4-phenyl-5,5-di-(2-methoxyphenyl)-2-oxazoline].

Exemplary salicylaldimine compounds include, but are not limited to, (R)—N-salicylidene-2-amino-1,1-diphenyl-1-propanol, (R)—N-(5-nitrosalicylidene)-2-amino-1,1-diphenyl-1-propanol, (R)—N-(3,5-dinitrosalicylidene)-2-amino-1,1-diphenyl-1-propoanol, (R)—N-(5-chlorosalicylidene)-2-amino-1,1-diphenyl-1-propanol, (R)-(3,5-dichlorosalicylidene)-2-amino-1,1-diphenyl-1-propanol, (R)—N-(3-fluorosalicylidene)-2-amino-1,1-diphenyl-1-propanol, (R)—N-(3-bromosalicylidene)-2-amino-1,1-diphenyl-1-propanol, (R)—N-(3-methylsalicylidene)-2-amino-1,1-diphenyl-1-propanol, (R)—N-(3-trifluoromethylsalicylidene)-2-amino-1,1-diphenyl-1-propanol, (R)—N-(5-trifluoromethylsalicylidene)-2-amino-1,1-diphenyl-1-propanol, (R)—N-(3-methoxysalicylidene)-2-amino-1,1-diphenyl-1-propanol, (R)—N-salicylidene-2-amino-1,1-di(2-methoxyphenyl)-1-propanol, (R)—N-(5-nitrosalicylidene)-2-amino-1,1-di(2-methoxyphenyl)-1-propanol, (R)—N-(5-chlorosalicylidene)-2-amino-1,1-di(2-methoxy-phenyl)-1-propanol, (R)—N-(3,5-dinitrosalicylidene)-2-amino-1,1-di(2-methoxy-phenyl)-1-propanol, (R)—N-(3,5-dichloro salicylidene)-2-amino-1,1-di(2-methoxy-phenyl)-1-propanol, (R)—N-(3-fluorosalicylidene)-2-amino-1,1-di(2-methoxyphenyl)-1-propanol, (R)—N-(3-bromosalicylidene)-2-amino-1,1-di(2-methoxyphenyl)-1-propanol, (R)—N-(3-methylsalicylidene)-2-amino-1,1-di(2-methoxyphenyl)-1-propanol, (R)—N-(3-trifluoromethylsalicylidene)-2-amino-1,1-di(2-methoxyphenyl)-1-propanol, (R)—N-(5-trifluoromethylsalicylidene)-2-amino-1,1-di(2-methoxyphenyl)-1-propanol, (R)—N-(3-methoxysalicylidene)-2-amino-1,1-di(2-methoxy-phenyl)-1-propanol, (R)—N-salicylidene-2-amino-1,1-di(2-n-butoxy-5-tert-butyl-phenyl)-1-propanol, (R)—N-(5-nitrosalicylidene)-2-amino-1,1-di(2-n-butoxy-5-tert-butylphenyl)-1-propanol, (R)—N-(3,5-dinitrosalicylidene)-2-amino-1,1-di(2-n-butoxy-5-tert-butylphenyl)-1-propanol, (R)—N-(5-chlorosalicylidene)-2-amino-1,1-di(2-n-butoxy-5-tert-butylphenyl)-1-propanol, (R)—N-(3,5-dichlorosalicylidene)-2-amino-1,1-di(2-n-butoxy-5-tert-butylphenyl)-1-propanol, (R)—N-(3-fluorosalicylidene)-2-amino-1,1-di(2-n-butoxy-5-tert-butylphenyl)-1-propanol, (R)—N-(3-bromosalicylidene)-2-amino-1,1-di(2-n-butoxy-5-tert-butylphenyl)-1-propanol, (R)—N-(3-methylsalicylidene)-2-amino-1,1-di(2-n-butoxy-5-tert-butyl phenyl)-1-propanol, (R)—N-(3-trifluoromethylsalicylidene)-2-amino-1,1-di(2-n-butoxy-5-tert-butylphenyl)-1-propanol, (R)—N-(5-trifluoromethylsalicylidene)-2-amino-1,1-di(2-n-butoxy-5-tert-butylphenyl)-1-propanol, (R)—N-(3-methoxysalicylidene)-2-amino-1,1-di(2-n-butoxy-5-tert-butylphenyl)-1-propanol, (R)—N-(5-methoxycarbonylsalicylidene)-2-amino-1,1-diphenyl-1-propanol, (R)—N-(2-hydroxy-1-naphtylidene)-2-amino-1,1-diphenyl-1-propanol, (R)—N-(1-hydroxy-2-naphtylidene)-2-amino-1,1-diphenyl-1-propanol and the like, and compounds having (S)-configuration in place of (R)-configuration in the above exemplified compounds.

Exemplary salen compounds include, but are not limited to, (1R,2R) or (1S,2S) isomers of 1,2-cyclo-hexanediamino-N,N′-bis-3,5-di-t-butylsalicylidene, 1,2-cyclohexanediamino-N,N′-bis-3,5-diiodosalicylidene, 1,2-phenylenediamino-N,N′-bis-3,5-di-t-butylsalicylidene, 4,5-dichloro-1,2-phenylenediamino-N,N′-bis-3,5-di-t-butylsalicylidene, 1,2-phenylenediamino-N,N′-bis-3,5-dimethoxysalicylidene, 1,2-(1,3,5-trimethylphenylene)diamino-N,N′-bis-3,5-di-t-butyl salicylidene, and mixtures thereof.

Exemplary Schiff bases include, but are not limited to, (1R,2S)-[1-[(3,5-di-tert-butyl-2-hydroxybenzylidene)amino]indan-2-ol], (1R,2S)-[1-[(3-adamantyl-2-hydroxy-5-methyl benzylidene)amino]indan-2-ol], (1S,2R)-[1-[(3-adamantyl-2-hydroxy-5-methylbenzylidene)amino]indan-2-ol], and (1R,2S)-[1-[(3-adamantyl-2-hydroxy-5-methylbenzylidene)amino]-1,2-d]-phenylethan-2-ol.

In one embodiment, the cyclopropanation reaction in step-(b) is carried out at a temperature of about 0° C. to about 100° C. for at least 30 minutes, specifically at a temperature of about 30° C. to about 70° C. for about 1 hour to about 5 hours, and more specifically at a temperature of about 45° C. to about 55° C. for about 2 hours to about 3 hours. In another embodiment, slower addition of the compounds of formulae V and VI is employed to obtain the compound of formula IV with higher level of enantiomeric excess. The preferred addition time of these compounds is 5 hours to 16 hours, more preferably 7 hours to 10 hours. In another embodiment, the reaction mass may be quenched into water after completion of the reaction.

The reaction mass containing the substituted cyclopropanecarboxylate compound of formula IV obtained in step-(b) may be subjected to usual work up such as a washing, an extraction, a pH adjustment, an evaporation or a combination thereof. The reaction mass may be used directly in the next step to produce the cyclopropanecarboxylic acid compound of formula III, or the cyclopropanecarboxylate compound of formula IV may be isolated and then used in the next step.

In one embodiment, the cyclopropanecarboxylate compound of formula IV is isolated from a suitable solvent by the methods as described above.

In another embodiment, the solvent used to isolate the cyclopropanecarboxylate compound of formula IV is selected from the group consisting of water, an aliphatic ether, a hydrocarbon solvent, a chlorinated hydrocarbon, and mixtures thereof. Specifically, the solvent is selected from the group consisting of water, toluene, xylene, dichloromethane, diethyl ether, diisopropyl ether, n-heptane, n-pentane, n-hexane, cyclohexane, and mixtures thereof.

In another embodiment, the reaction mass containing the cyclopropanecarboxylate compound of formula IV obtained is concentrated and then taken for next step.

Exemplary acids used in step-(c) include, but are not limited to, methanesulfonic acid, trifluoromethanesulfonic acid, trifluoroacetic acid, hydrochloric acid, sulfuric acid and the like, and mixtures thereof.

Exemplary second bases used in step-(c) include, but are not limited to, sodium hydroxide, potassium hydroxide, lithium hydroxide, calcium hydroxide, magnesium hydroxide, tetra-n-butyl ammonium hydroxide, and mixtures thereof. A most specific base is sodium hydroxide.

Exemplary third solvents used in step-(c) include, but are not limited to, water, an alcohol, a ketone, a cyclic ether, an aliphatic ether, a hydrocarbon, a chlorinated hydrocarbon, a nitrile, and mixtures thereof. The term solvent also includes mixtures of solvents.

In one embodiment, the third solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, dichloromethane, dichloroethane, chloroform, carbon tetrachloride, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, and mixtures thereof; more specifically, the third solvent is selected from the group consisting of water, methanol, ethanol, n-propanol, isopropanol, and mixtures thereof; and most specifically methanol.

In one embodiment, the hydrolysis reaction in step-(c) is carried out at a temperature of about 0° C. to about 100° C. for at least 30 minutes, specifically at a temperature of about 30° C. to about 80° C. for about 1 hour to about 6 hours, and more specifically at a temperature of about 45° C. to about 65° C. for 2 hours to about 4 hours.

The reaction mass containing the substituted cyclopropanecarboxylic acid compound of formula III obtained in step-(c) may be subjected to usual work up by the methods as described above. The reaction mass may be used directly in the next step to produce the substituted phenylcyclopropylamine compound of formula II, or the cyclopropanecarboxylic acid compound of formula III may be isolated and/or purified and then used in the next step.

In one embodiment, the reaction mass containing the substituted cyclopropanecarboxylic acid compound of formula III is converted to its amine salt by treating with a suitable chiral amine, followed by acidification with a suitable acid to produce pure compound of formula III.

Exemplary chiral amines (and their isomers) used in step-(d) include, but are not limited to, (S)-(−)-methylbenzylamine, (+)-dehydroabietylamine, (−)-(α)-N-benzylphenethylamine, (−)-(α)-methylbenzylamine, (−)-2-aminobutanol, (−)-brucine, (−)-cinchonine, (−)-dehydroabietylamine, (−)-quinine, (−)-ephedrine, (−)-substituted phenyl glycinol, (1S,2R)-(−)-cis-1-amino-2-indanol, (R)-(−)-aminoindane, (−)-2-amino-1-hexanol, (−)-α-tolylethyl amine, (−)-3-methyl-2-phenylbutylamine, (1R,2S)-(−)-2-amino-1,2-diphenylethanol, D-(−)-threo-2-amino-1-(4-nitrophenyl)-1,3-propanediol, D-(−)-arginine, (−)-cis-2-benzylaminocyclohexanemethanol, L-(+)-lysinemonohydrochloride, (s)-α-methyl-4-nitrobenzylaminehydrochloride, (S)-(−)-1-(1-naphthyl)ethylamine, L-phenylalaminol, (S)-1-phenyl-2-(p-tolyl)ethyl amine, strychnine, (S)-(−)-1-(p-tolyl)ethylamine, (−)-(α)-phenylethanesulfonic acid, (R)-(−)-amphetamine, N-alkyl-D-glucamines, and mixtures thereof. A most specific chiral amine is (S)-(−)-methylbenzylamine.

Exemplary fourth solvents used in step-(d) include, but are not limited to, water, an alcohol, a ketone, a cyclic ether, an aliphatic ether, a hydrocarbon, a chlorinated hydrocarbon, a nitrile, and mixtures thereof.

In one embodiment, the fourth solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, dichloromethane, dichloroethane, chloroform, carbon tetrachloride, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, and mixtures thereof; more specifically, the fourth solvent is selected from the group consisting of water, methanol, ethanol, n-propanol, isopropanol, and mixtures thereof; and most specifically isopropanol.

The amine salt of cyclopropanecarboxylic acid compound of formula III obtained in step-(d) may be used directly in the next step to produce the substituted phenylcyclopropylamine compound of formula II, or the cyclopropanecarboxylic acid compound of formula III may be acidified with an acid to produce a free acid and then used in the next step.

Exemplary fifth solvents used in step-(f) include, but are not limited to, a ketone, an ester, a hydrocarbon, a chlorinated hydrocarbon, a cyclic ether, an aliphatic ether, and mixtures thereof. The term solvent also includes mixtures of solvents.

In one embodiment, the fifth solvent is selected from the group consisting of acetone, methyl ethyl ketone, methyl isobutyl ketone, methyl tert-butyl ketone, ethyl acetate, methyl acetate, isopropyl acetate, tert-butyl methyl acetate, ethyl formate, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, dichloromethane, dichloroethane, chloroform, carbon tetrachloride, and mixtures thereof; and most specifically toluene, tetrahydrofuran, 2-methyl tetrahydrofuran, and mixtures thereof.

Exemplary third bases suitable for facilitating the rearrangement reaction in step-(f) include, but are not limited to, organic amine bases as described above. Specific bases are trimethylamine, dimethyl amine, diethylamine, tert-butyl amine, tributylamine, triethylamine, diisopropylethylamine, pyridine, N-methylmorpholine, 4-(N,N-dimethylamino)pyridine, 1,8-diazabicyclo[5.4.0]undec-7-ene; and most specifically triethylamine, diisopropylethylamine and 1,8-diazabicyclo[5.4.0]undec-7-ene.

Exemplary azides used in step-(f) include, but are not limited to, diethylphosphoryl azide, diisopropylphosphoryl azide, di-tert-butylphosphoryl azide, dibutylphosphoryl azide, dibenzylphosphoryl azide, di-1 or d-menthylphosphoryl azide, diphenylphosphoryl azide, and mixtures thereof.

In one embodiment, the rearrangement reaction in step-(f) is carried out at a temperature of about 80° C. to about 150° C. for at least 20 minutes, specifically at a temperature of about 100° C. to about 130° C. for about 30 minutes to about 5 hours, and more specifically at a about 110° C. to about 120° C. for about 1 hour to about 4 hours.

The reaction mass may be evaporated to obtain crude isocyanate, which may be used directly to produce substituted phenylcyclopropylamine derivatives of formula II by subjecting the isocyante intermediate to acidic hydrolysis.

Exemplary acids used for facilitating the hydrolysis of isocyanate intermediate in step-(f) include, but are not limited to, methanesulfonic acid, trifluoromethanesulfonic acid, trifluoroacetic acid, hydrochloric acid, sulfuric acid, and mixture thereof.

Exemplary sixth solvents used for hydrolysis in step-(f) include, but are not limited to, water, a ketone, an ester, a hydrocarbon, a chlorinated hydrocarbon, a cyclic ether, an aliphatic ether, and mixtures thereof.

In one embodiment, the sixth solvent is selected from the group consisting of water, acetone, methyl ethyl ketone, methyl isobutyl ketone, methyl tert-butyl ketone, ethyl acetate, methyl acetate, isopropyl acetate, tert-butyl methyl acetate, ethyl formate, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, dichloromethane, dichloroethane, chloroform, carbon tetrachloride, and mixtures thereof; and most specifically water, dioxane, tetrahydrofuran, 2-methyl tetrahydrofuran, and mixtures thereof.

In one embodiment, the isocyanate hydrolysis in step-(f) is carried out at a temperature of about 20° C. to about 80° C. for at least 30 minutes, specifically at a temperature of about 30° C. to about 70° C. for about 1 hour to about 4 hours, and more specifically at about 40° C. to about 50° C. for about 2 hours to about 3 hours.

The reaction mass containing the substituted phenylcyclopropylamine derivatives of formula II or a stereochemically isomeric form or a mixture of stereochemically isomeric forms thereof obtained in step-(f) may be subjected to usual work up, and followed by isolating and/or recovering from a suitable solvent by the methods as described above.

In one embodiment, the substituted phenylcyclopropylamine derivatives of formula II or a stereochemically isomeric form or a mixture of stereochemically isomeric forms thereof obtained in step-(f) is subjected to usual work up and then recovered by techniques such as filtration, filtration under vacuum, decantation, centrifugation, or a combination thereof. In another embodiment, the compound of formula II is recovered by filtration employing a filtration media of, for example, a silica gel or celite.

In another embodiment, the acidic reaction mixture obtained in step-(f) may be washed with water immiscible solvents to separate impurities from desired amine compound. Exemplary water immiscible solvents used for washing include, but are not limited to, isopropyl acetate, isobutyl acetate, tert-butyl acetate, diisopropyl ether, methyl tert-butyl ether, monoglyme, diglyme, cyclohexane, toluene, xylene, and mixtures thereof.

In one embodiment, the product is recovered from aqueous medium after basification with the fourth base, wherein the fourth base is selected from the group containing organic and inorganic bases as described above.

Specific fourth bases are aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide, and more specifically sodium hydroxide.

The use of inexpensive, non-explosive, non-hazardous, readily available and easy to handle reagents and solvents allows the process disclosed herein to be suitable for preparation of the substituted phenylcyclopropylamine derivatives of formula II or a stereochemically isomeric form or a mixture of stereochemically isomeric forms thereof at lab scale and in commercial scale operations.

Acid addition salts of the compounds of formula II can be prepared in high purity by using the substantially pure substituted phenylcyclopropylamine derivatives of formula II or a stereochemically isomeric form or a mixture of stereochemically isomeric forms thereof obtained by the method disclosed herein, by known methods.

The acid addition salts of substituted phenylcyclopropylamine derivatives of formula II or a stereochemically isomeric form or a mixture of stereochemically isomeric forms thereof are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, (1S)-(+)-10-camphorsulfonic acid, (R) or (S)-α-methoxy-α-(trifluoromethyl)-phenylacetic acid (Mosher's acid), (S) or (R)-(−)-(2-phenylcarbamoyloxy)propionic acid [(S)-(−)-carbamalactic acid], (R) or (S)-para-methylmandelic acid, (R) or (S)-ortho-chloromandelic acid, (R) or (S)-2-hydroxymethylhexanoic acid, (R) or (S)-2-hydroxymethylbutanoic acid, and (R) or (S)-2-hydroxymethylpropanoic acid.

Specific acid addition salts of the compounds of formula II are tartrate, di-p-toluoyl-tartrate, (S)-ketopinate, (D)-malate, (D)-camphorsulfonate, (R)-(−)-α-methoxyphenyl acetate, fumarate, phosphate and sulfate salts.

The term “substantially pure substituted phenylcyclopropylamine derivatives” refers to the substituted phenylcyclopropylamine derivatives having a total purity, including both stereochemical and chemical purity, of greater than about 95%, specifically greater than about 98%, more specifically greater than about 99%, and still more specifically greater than about 99.5%. The purity is preferably measured by High Performance Liquid Chromatography (HPLC). For example, the purity of the substituted phenylcyclopropylamine derivatives obtained by the process disclosed herein is about 95% to about 99%, or about 98% to about 99.5%, as measured by HPLC.

According to another aspect, there is provided a process for preparing substituted phenylcyclopropylamine derivatives of formula II:

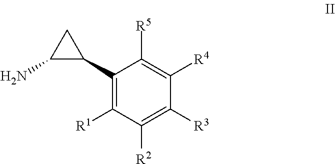

or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I, preferably, the halogen atom is F; comprising:
a) reacting the substituted cyclopropanecarboxylic acid compound of formula III:

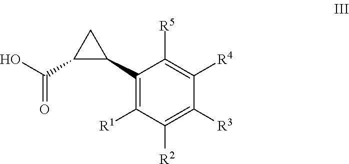

or a stereochemically isomeric form or a mixture of stereochemically isomeric forms thereof, or an acid addition salt thereof, wherein R¹, R², R³, R⁴ and R⁵ are as defined in formula II; with an azide compound, with the proviso that the azide does not include sodium azide, in the presence of an alcohol and a base, optionally in the presence of a first solvent, to produce a substituted cyclopropanecarbamate compound of formula IX:

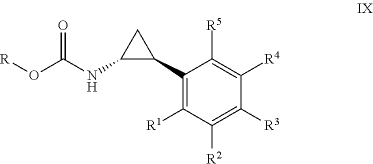

or a stereochemically isomeric form or a mixture of stereochemically isomeric forms thereof, wherein ‘R’ is an alkyl, cycloalkyl, aryl or aralkyl group; and wherein R¹, R², R³, R⁴ and R⁵ are as defined in formula II; and
b) subjecting the cyclopropanecarbamate compound of formula IX to acidic hydrolysis with an acid in a second solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.

Exemplary alcohols used in step-(a) include, but are not limited to, C₁₋₆ straight or branched chain alcohols, cycloalkanols and aromatic alcohols. In one embodiment, the alcohol is selected from the group consisting of methanol, ethanol, isopropyl alcohol, isobutanol, tert-butanol, n-pentanol, cyclohexanol, 1 or d-menthol, benzyl alcohol, and mixtures thereof.

In one embodiment, the alcohol in step-(a) is used in a molar equivalent or in excess or used as a solvent medium. The reaction may be carried out in the presence of a reaction inert solvent when the alcohol is used in an amount of molar equivalent.

Exemplary first solvents used in step-(a) include, but are not limited to, an ester, a nitrile, a hydrocarbon, a cyclic ether, an aliphatic ether, and mixtures thereof. The term solvent also includes mixtures of solvents.

Specifically, the first solvent is selected from the group consisting of ethyl acetate, isopropyl acetate, isobutyl acetate, tert-butyl acetate, acetonitrile, propionitrile, tetrahydrofuran, 2-methyl tetrahydrofuran, 1,4-dioxane, methyl tert-butyl ether, diethyl ether, diisopropyl ether, monoglyme, diglyme, n-hexane, n-heptane, cyclohexane, toluene, xylene, and mixtures thereof; and most specifically toluene, tetrahydrofuran, 2-methyl tetrahydrofuran, and mixtures thereof.

Exemplary bases suitable for facilitating the rearrangement reaction in step-(a) include, but are not limited to, organic amine bases as described above. Specific bases are trimethylamine, dimethylamine, diethylamine, tert-butyl amine, tributylamine, triethylamine, diisopropylethyl amine, pyridine, N-methylmorpholine, 4-(N,N-dimethylamino)pyridine, 1,8-diazabicyclo[5.4.0]undec-7-ene; and most specifically triethylamine, diisopropylethylamine and 1,8-diazabicyclo[5.4.0]undec-7-ene.

Exemplary azides used in step-(a) include, but are not limited to, diethylphosphoryl azide, diisopropylphosphoryl azide, di-tert-butylphosphoryl azide, dibutylphosphoryl azide, dibenzylphosphoryl azide, di-1 or d-menthylphosphoryl azide, diphenylphosphoryl azide, and mixtures thereof.

In one embodiment, the rearrangement reaction in step-(a) is carried out at a temperature of about 50° C. to the boiling temperature of the solvent used for at least 2 hours, specifically at a temperature of about 80° C. to the boiling temperature of the solvent used for about 5 hours to about 24 hours, and more specifically at the boiling temperature of the solvent for about 14 hours to about 18 hours.

The reaction mass containing the substituted cyclopropanecarbamate compound of formula IX obtained in step-(a) may be used directly in the next step or the carbamate compound may be recovered from the reaction medium by customary work-up and then used in the next step.

Exemplary acids used in step-(b) for carbamate hydrolysis include, but are not limited to methanesulfonic acid, trifluoromethanesulfonic acid, trifluoroacetic acid, hydrochloric acid, sulfuric acid, hydrobromic acid, and mixtures thereof.

Exemplary second solvents used in step-(b) include, but are not limited to, water, an alcohol, an ester, a cyclic ether, an aliphatic ether, a hydrocarbon, and mixtures thereof.

In one embodiment, the second solvent is selected from the group consisting of water, methanol, ethanol, n-propanol, isopropanol, n-butanol, isobutanol, tert-butanol, amyl alcohol, ethyl acetate, isopropyl acetate, isobutyl acetate, tert-butyl acetate, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, and mixtures thereof; and more specifically, the second solvent is selected from the group consisting of water, tetrahydrofuran, 2-methyl tetrahydrofuran, dioxane, and mixtures thereof.

In one embodiment, the carbamate hydrolysis in step-(b) is carried out at a temperature of about 20° C. to about 80° C. for at least 30 minutes, specifically at a temperature of about 30° C. to about 70° C. for about 2 hours to about 10 hours, and more specifically at a temperature of about 40° C. to about 50° C. for about 4 hours to about 8 hours.

The reaction mass containing the substituted phenylcyclopropylamine derivatives of formula II or a stereochemically isomeric form or a mixture of stereochemically isomeric forms thereof obtained in step-(b) may be subjected to usual work up methods, followed by isolating and/or recovering from a suitable solvent by the methods as described above.

In one embodiment, the acidic reaction mixture obtained in step-(b) is washed with a water immiscible solvent to separate impurities from desired amine compound. Exemplary water immiscible solvents used for washing include, but are not limited to, isopropyl acetate, isobutyl acetate, tert-butyl acetate, diisopropyl ether, methyl tert-butyl ether, monoglyme, diglyme, cyclohexane, toluene, xylene, and mixtures thereof.

In another embodiment, the phenylcyclopropylamine derivatives of formula II are recovered from the aqueous medium after basification with a suitable base, wherein the base is selected from the group consisting of organic and inorganic bases as described above.

According to another aspect, there is provided a process for preparing substituted phenylcyclopropylamine derivatives of formula II:

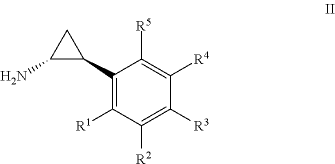

or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I, preferably, the halogen atom is F; comprising:
a) reacting the substituted cyclopropanecarboxylic acid compound of formula III:

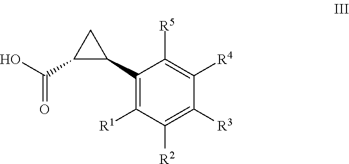

or a stereochemically isomeric form or a mixture of stereochemically isomeric forms thereof, or an amine salt thereof, wherein R¹, R², R³, R⁴ and R⁵ are as defined in formula II; with an activating agent in the presence of a base, optionally in the presence of a racemisation suppressant, in a first solvent to produce an intermediate compound, followed by amidation with hydroxyl amine or an acid addition salt thereof to produce a cyclopropanecarboxamide compound of formula X:

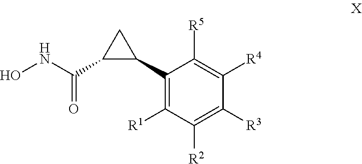

or a stereochemically isomeric form or a mixture of stereochemically isomeric forms thereof, wherein R¹, R², R³, R⁴ and R⁵ are as defined above;
b) reacting the cyclopropanecarboxamide compound of formula X with an activating agent, followed by treatment with an alcohol, optionally in the presence of a second solvent, to produce a substituted cyclopropanecarbamate compound of formula IX:

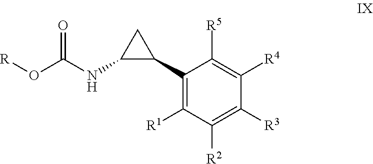

or a stereochemically isomeric form or a mixture of stereochemically isomeric forms thereof, wherein ‘R’ is an alkyl, cycloalkyl, aryl or aralkyl group; and wherein R¹, R², R³, R⁴ and R⁵ are as defined in formula II; and
c) subjecting the cyclopropanecarbamate compound of formula IX to acidic hydrolysis with an acid in a third solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.

Exemplary first solvents used in step-(a) include, but are not limited to, water, a ketone, an ester, a hydrocarbon, a chlorinated hydrocarbon, a cyclic ether, an aliphatic ether, a nitrile, a polar aprotic solvent, and mixtures thereof.

In one embodiment, the first solvent is selected from the group consisting of water, acetone, methyl ethyl ketone, methyl isobutyl ketone, methyl tert-butyl ketone, cyclopentanone, ethyl acetate, methyl acetate, isopropyl acetate, tert-butyl methyl acetate, ethyl formate, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, dichloromethane, dichloroethane, chloroform, carbon tetrachloride, acetonitrile, propionitrile, 4-methylmorpholine, N,N-dimethylacetamide, nitromethane, triethylamine, N-methylpyrrolidone, and mixtures thereof; and more specifically, the first solvent is selected from the group consisting of acetone, dioxan, ethyl acetate, mixtures of ortho-xylene, meta-xylene, para-xylene, toluene, acetonitrile, tetrahydrofuran, dichloromethane, chloroform, methylethylketone, and mixtures thereof.

In one embodiment, the base used in step-(a) is an organic or inorganic base selected from the group as described above. Specific bases are aqueous ammonia, sodium hydroxide, potassium hydroxide, lithium hydroxide, sodium carbonate, potassium carbonate, sodium bicarbonate, potassium bicarbonate, trimethylamine, dimethylamine, diethylamine, tert-butyl amine, tributylamine, triethylamine, diisopropylethyl amine, pyridine, N-methylmorpholine, 4-(N,N-dimethylamino)pyridine, 1,8-diazabicyclo[5.4.0]undec-7-ene.

Exemplary activating agents used in step-(a) include, but are not limited to, 1,1-carbonyldiimidazole, 1,1′-carbonyl-di-(1,2,4-triazole), phosgene derivatives, alkyl chloroformates, arylchloro formates, 2-halo-4,6-dialkoxy-1,3,5-triazines, thionyl chloride, trialkyl phosphites, triarylphosphites, N,N-dialkylcarbodiimides, N,N-diarylcarbodiimides, diphenylphosphorylazide, 1-chloro-N,N,2-trimethyl-1-propenyl amine, chloro-N,N,N′,N′-bis(tetra-ethylene)formamidinium tetrafluoro borate, boric acid derivatives, fluoro-N,N,N′,N′-bis(tetramethylene)formamidiniumhexafluorophosphates, oxalic acid diimidazole, 2-halo-1,3-dimethylimidazo lidinium chloride, 2-halo-1,3-dimethylimidazo lidinium hexafluorophosphate, benzotriazole-phosphonium salt complexes, pyrrolidinephosphoniumsalts, 3-(diethoxyphosphoryloxy)-1,2,3-benzotriazin-4(3H)-one, N/O-substituted benzotriazole salts/derivatives, O-(2-oxo-1(2H)pyridyl)-N,N,N′,N′-tetramethyluronium tetrafluoroborate, O-[(ethoxycarbonyl)cyanomethylenamino]-N,N,N′,N′-tetramethyluronium hexafluorophosphate (HOTU), O-[(ethoxycarbonyl)cyanomethylenamino]-N,N,N′,N′-tetramethyluronium tetrafluoroborate (TOTU) and other uronium complexes, polyphosphonic anhydride, thiouronium reagents, and mixtures thereof.

Exemplary racemisation suppressants used in step-(a) includes, but are not limited to, 1-hydroxybenzotriazole, 1-hydroxy-7-azabenzotriazole, ethyl-1-hydroxy-1H-1,2,3-triazole carboxylate, N-hydroxytetrazole, 1-hydroxy-substitutedtetrazoles, 1-hydroxy-substitutedbenzo-triazines, arylphosphonium salts, and mixtures thereof. A specific racemisation suppressant is 1-hydroxybenzotriazole.

In one embodiment, the acid activation reaction in step-(a) is carried out at a temperature of about −50° C. to about 30° C. for about 1 hour to about 20 hours, specifically at a temperature of about −30° C. to about 20° C. for about 2 hours to about 18 hours, and more specifically at a temperature of about 0° C. to about 10° C. for about 2 hours to about 5 hours.

The hydroxyl amine in step-(a) may be used, in the form a solid or a solution, as a base or a salt of hydroxyl amine. In one embodiment, the salt of hydroxyl amine is basified in-situ using a suitable base.

In one embodiment, the amidation reaction step-(a) is carried out at a temperature of about −50° C. to about 50° C. for about 1 hour to about 20 hours, specifically at a temperature of about −30° C. to about 40° C. for about 2 hours to about 18 hours, and more specifically at a temperature of about 0° C. to about 30° C. for about 2 hours to about 5 hours.

The reaction mass containing the substituted cyclopropanecarboxamide compound of formula X obtained in step-(a) may be used directly in the next step or the carboxamide compound may be recovered from the reaction medium by customary work-up and then used in the next step.

Exemplary alcohols used in step-(b) include, but are not limited to, C₁₋₆ straight or branched chain alcohols, cycloalkanols and aromatic alcohols. In one embodiment, the alcohol is selected from the group consisting of methanol, ethanol, isopropyl alcohol, isobutanol, tert-butanol, n-pentanol, cyclohexanol, 1 or d-menthol, benzyl alcohol, and mixtures thereof.

In one embodiment, the alcohol in step-(b) is used in a molar equivalent or in excess or used as a solvent media. The reaction may be carried out in the presence of a reaction inert solvent incase the alcohol is used in an amount of molar equivalent.

Exemplary second solvents used in step-(b) include, but are not limited to, an ester, a nitrile, a hydrocarbon, a cyclic ether, an aliphatic ether, and mixtures thereof. The term solvent also includes mixtures of solvents.

Specifically, the second solvent is selected from the group consisting of ethyl acetate, isopropyl acetate, isobutyl acetate, tert-butyl acetate, acetonitrile, propionitrile, tetrahydrofuran, 2-methyl tetrahydrofuran, 1,4-dioxane, methyl tert-butyl ether, diethyl ether, diisopropyl ether, monoglyme, diglyme, n-hexane, n-heptane, cyclohexane, toluene, xylene, and mixtures thereof; and most specifically toluene, tetrahydrofuran, 2-methyl tetrahydrofuran, and mixtures thereof.

In one embodiment, the activating agent used in step-(b) is selected from the group as described above. A specific activating agent is 1,1-carbonyldiimidazole.

In another embodiment, the reaction in step-(a) is carried out at the boiling temperature of the solvent used. The reaction time may vary from about 5 hours to about 24 hours, specifically from about 10 hours to about 20 hours, and more specifically from about 14 hours to about 18 hours.

The reaction mass containing the substituted cyclopropanecarbamate compound of formula IX obtained in step-(b) may be used directly in the next step or the carbamate compound may be recovered from the reaction medium by customary work-up and then used in the next step.

The conversion of the cyclopropanecarbamate compound of formula IX to the phenylcyclopropylamine derivatives of formula II in step-(c) is carried out by the methods as described herein above.

According to another aspect, there is provided a one pot process for preparing substituted phenylcyclopropylamine derivatives of formula II:

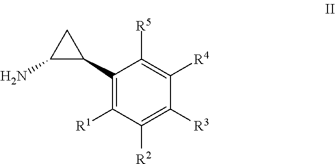

or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring is substituted with at least one or more halogen atoms, wherein the halogen atom is F, Cl, Br or I, preferably, the halogen atom is F; comprising:
a) reacting the substituted cyclopropanecarboxylic acid compound of formula III:

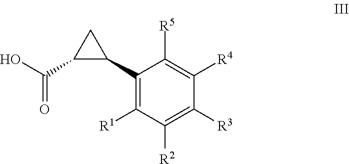

or a stereochemically isomeric form or a mixture of stereochemically isomeric forms thereof, or an amine salt thereof, wherein R¹, R², R³, R⁴ and R⁵ are as defined in formula II; with an acid activating agent in the presence of a base in a solvent to produce an intermediate compound, followed by amidation with hydroxyl amine or an acid addition salt thereof to produce a cyclopropanecarboxamide compound of formula X:

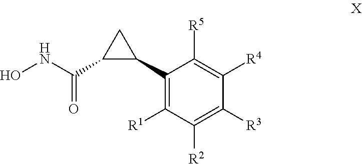

or a stereochemically isomeric form or a mixture of stereochemically isomeric forms thereof, wherein R¹, R², R³, R⁴ and R⁵ are as defined above;
b) reacting the cyclopropanecarboxamide compound of formula X, in-situ, with a carbonyl source to produce a cyclopropanedioxazol compound of formula XI:

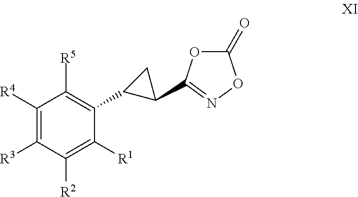

or a stereochemically isomeric form or a mixture of stereochemically isomeric forms thereof, wherein R¹, R², R³, R⁴ and R⁵ are as defined above;
c) subjecting the cyclopropanedioxazol compound of formula XI, in-situ, to thermal rearrangement at boiling temperature of the reaction solvent to produce a cyclopropaneisocyanate compound of formula XII:

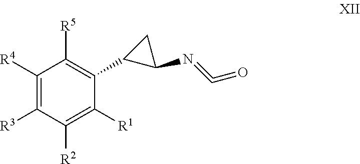

or a stereochemically isomeric form or a mixture of stereochemically isomeric forms thereof, wherein R¹, R², R³, R⁴ and R⁵ are as defined above;
d) reacting the cyclopropaneisocyanate compound of formula XII, in-situ, with an alcohol at the boiling temperature to produce a cyclopropanecarbamate compound of formula IX:

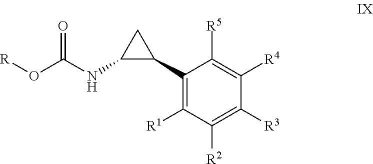

or a stereochemically isomeric form or a mixture of stereochemically isomeric forms thereof, wherein ‘R’ is an alkyl, cycloalkyl, aryl or aralkyl group; and wherein R¹, R², R³, R⁴ and R⁵ are as defined in formula II; and
e) subjecting the cyclopropanecarbamate compound of formula IX to acidic hydrolysis with an acid to produce the substituted phenylcyclopropylamine derivatives of formula II or a stereochemically isomeric form or a mixture of stereochemically isomeric forms thereof, and optionally converting the compound of formula II obtained into an acid addition salt thereof.

Exemplary solvents used in the above one pot process include, but are not limited to, water, a ketone, an ester, a hydrocarbon, a chlorinated hydrocarbon, a cyclic ether, an aliphatic ether, a nitrile, a polar aprotic solvent, and mixtures thereof.

In one embodiment, the solvent is selected from the group consisting of water, acetone, methyl ethyl ketone, methyl isobutyl ketone, methyl tert-butyl ketone, cyclopentanone, ethyl acetate, methyl acetate, isopropyl acetate, tert-butyl methyl acetate, ethyl formate, tetrahydrofuran, 2-methyl tetrahydrofuran, dioxane, diethyl ether, diisopropyl ether, methyl tert-butyl ether, monoglyme, diglyme, n-pentane, n-hexane, n-heptane, cyclohexane, toluene, xylene, dichloromethane, dichloroethane, chloroform, carbon tetrachloride, acetonitrile, propionitrile, 4-methylmorpholine, N,N-dimethylacetamide, nitromethane, triethylamine, N-methylpyrrolidone, and mixtures thereof; and more specifically, the solvent is selected from the group consisting of acetone, dioxan, ethyl acetate, mixtures of ortho-xylene, meta-xylene, para-xylene, toluene, acetonitrile, tetrahydrofuran, dichloromethane, chloroform, methylethylketone, and mixtures thereof.

The base used in the above one pot process is an organic or inorganic base selected from the group as described above.

The activating agents used for the one pot process can be selected from the group as described above.

The hydroxyl amine in step-(a) may be used, in the form a solid or a solution, as a base or a salt of hydroxyl amine. In one embodiment, the salt of hydroxyl amine is basified in-situ using a suitable base.

Exemplary carbonyl sources used in step-(b) include, but are not limited to, 1,1′-carbonyldiimidazole, 1,1′-carbonyl-di-(1,2,4-triazole), phosgene derivatives, alkyl chloroformates, arylchloroformates, and mixtures thereof. A specific carbonyl source is 1,1′-carbonyldiimidazole.

Exemplary alcohols used in step-(d) include, but are not limited to, C₁₋₆ straight or branched chain alcohols, cycloalkanols and aromatic alcohols. In one embodiment, the alcohol is selected from the group consisting of methanol, ethanol, isopropyl alcohol, isobutanol, tert-butanol, n-pentanol, cyclohexanol, 1 or d-menthol, benzyl alcohol, and mixtures thereof.

In one embodiment, the alcohol in step-(d) is used in a molar equivalent or in excess or used as a solvent media. The reaction may be carried out in presence of a reaction inert solvent when the alcohol is used in an amount of molar equivalent.

The overall one-pot process may carried out at a temperature of about −50° C. to about 150° C., specifically at a temperature of about −30° C. to about 140° C., and more specifically at a temperature of about 0° C. to about 100° C. The reaction time may vary from about 1 hour to about 25 hours, specifically from about 5 hours to about 20 hours, and more specifically from about 10 hours to about 15 hours.

The conversion of the cyclopropanecarbamate compound of formula IX to the phenylcyclopropylamine derivatives of formula II in step-(c) is carried out by the methods as described herein above.

Aptly the processes of this disclosure are adapted to the preparation of triazolo[4,5-d]pyrimidinecyclopentane compounds, preferably Ticagrelor, and their pharmaceutically acceptable acid addition salts, in high enantiomeric and chemical purity.

Ticagrelor and pharmaceutically acceptable acid addition salts of ticagrelor can be prepared in high purity by using the substantially pure trans-(1R,2S)-2-(3,4-difluorophenyl)-cyclopropylamine of formula IIa or an acid addition salt thereof obtained by the methods disclosed herein, by known methods.

The intermediate compounds of formulae IX, X, XI and XII, and their stereochemical isomers are novel and form another aspect of the present invention.

The use of the intermediate compounds of formulae V, VI, IX, X, XI and XII, and their stereochemical isomers, in the preparation of substituted phenylcyclopropylamine derivatives of formula II or a stereochemically isomeric form or a mixture of stereochemically isomeric forms thereof is novel and forms further aspect of the present invention.

Solid state forms of trans-(1R,2S)-2-(3,4-difluorophenyl)-cyclopropylamine acid addition salts, except the mandelate salt, have not been reported, isolated, or characterized in the literature. The present inventors have surprisingly and unexpectedly found that some of the acid addition salts of trans-(1R,2S)-2-(3,4-difluorophenyl)-cyclopropylamine, specifically, the tartrate salt, di-p-toluoyl-tartrate salt, (S)-ketopinate salt, (D)-malate salt, (D)-camphor sulfonate salt, (R)-(−)-α-methoxyphenyl acetate salt, fumarate salt, phosphate salt and sulfate salt, can be isolated as solid state forms.

It has also been found that the solid state forms of trans-(1R,2S)-2-(3,4-difluorophenyl)-cyclopropylamine acid addition salts are useful intermediates in the preparation of ticagrelor or a pharmaceutically acceptable salt thereof in high purity.

According to one aspect, provided herein are novel acid addition salts of trans-(1R,2S)-2-(3,4-difluorophenyl)-cyclopropylamine, wherein the acid addition salt is a tartrate salt, a di-p-toluoyl-tartrate salt, an (S)-ketopinate salt, a (D)-malate salt, a (D)-camphorsulfonate salt, a (R)-(−)-α-methoxyphenyl acetate salt, a fumarate salt, a phosphate salt or a sulfate salt.

In one embodiment, the acid addition salts of trans-(1R,2S)-2-(3,4-difluorophenyl)-cyclopropylamine in a solid state form are provided. In another embodiment, the solid state forms of trans-(1R,2S)-2-(3,4-difluorophenyl)-cyclopropylamine salts exist in a crystalline form. In another embodiment, the solid state forms of trans-(1R,2S)-2-(3,4-difluorophenyl)-cyclopropylamine salts exist in an amorphous form.

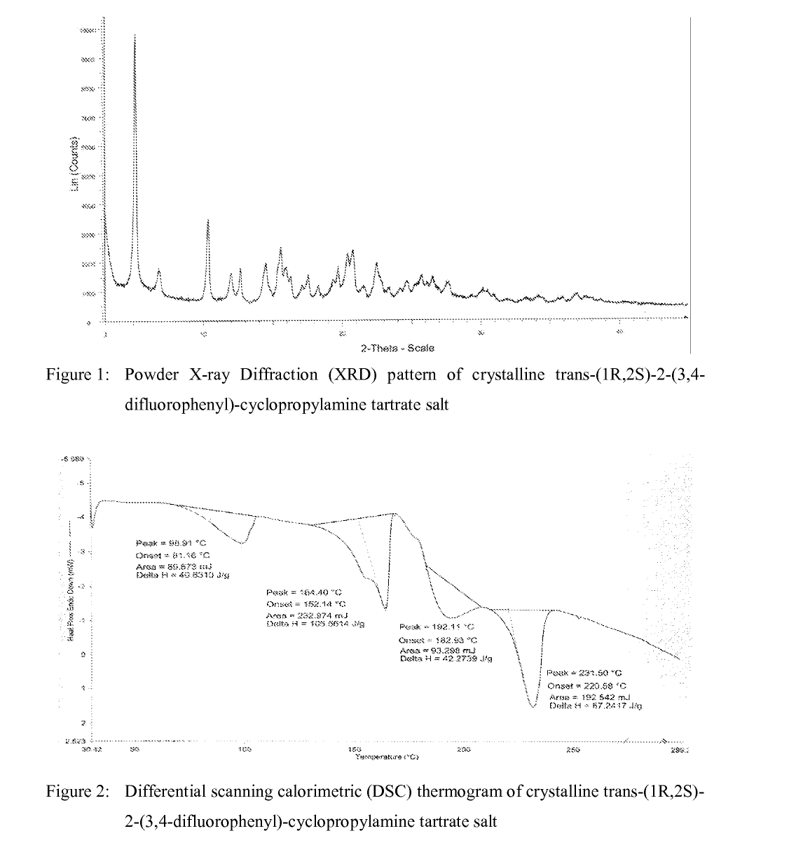

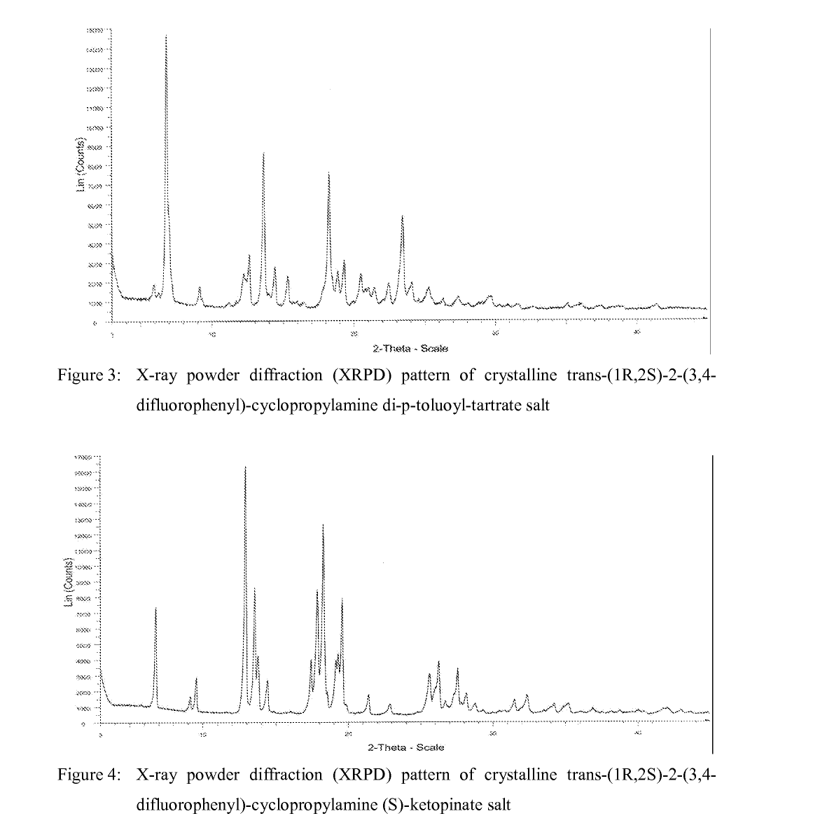

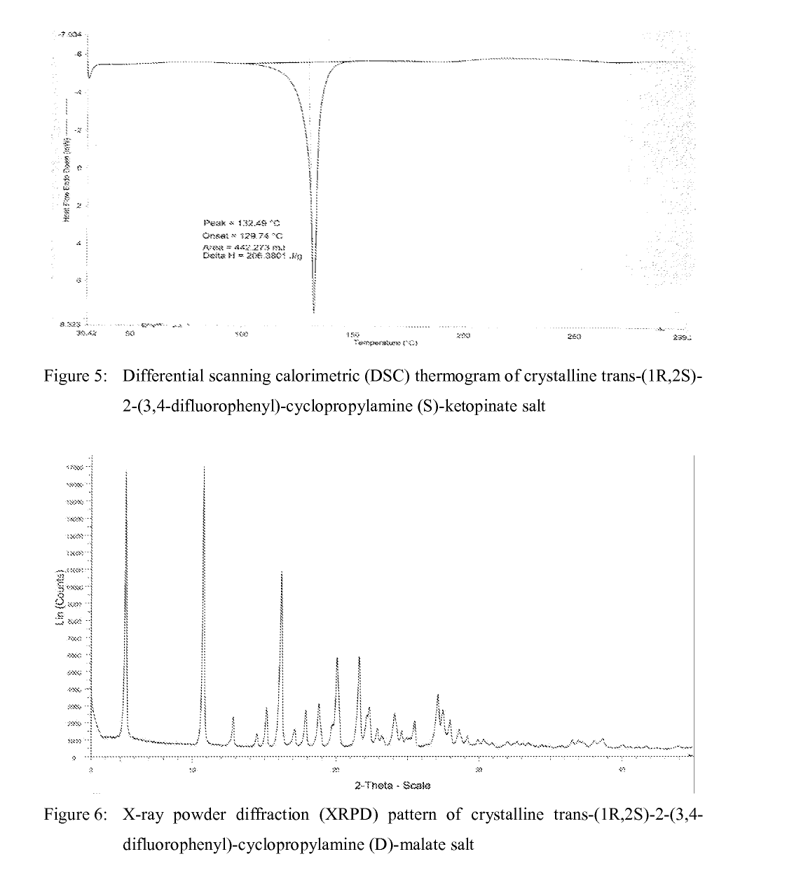

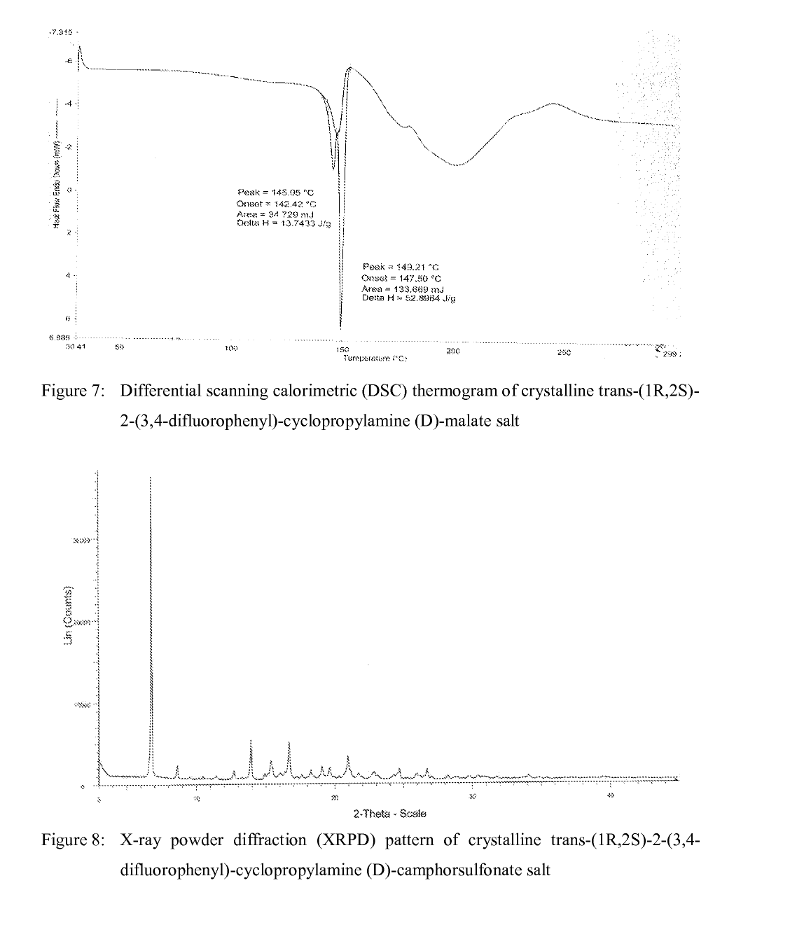

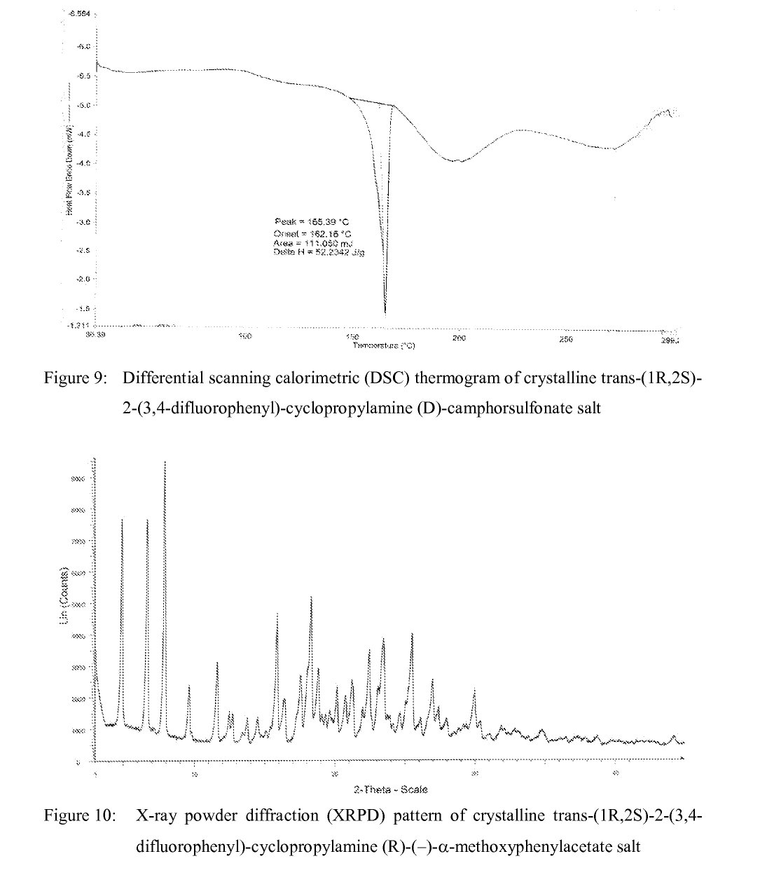

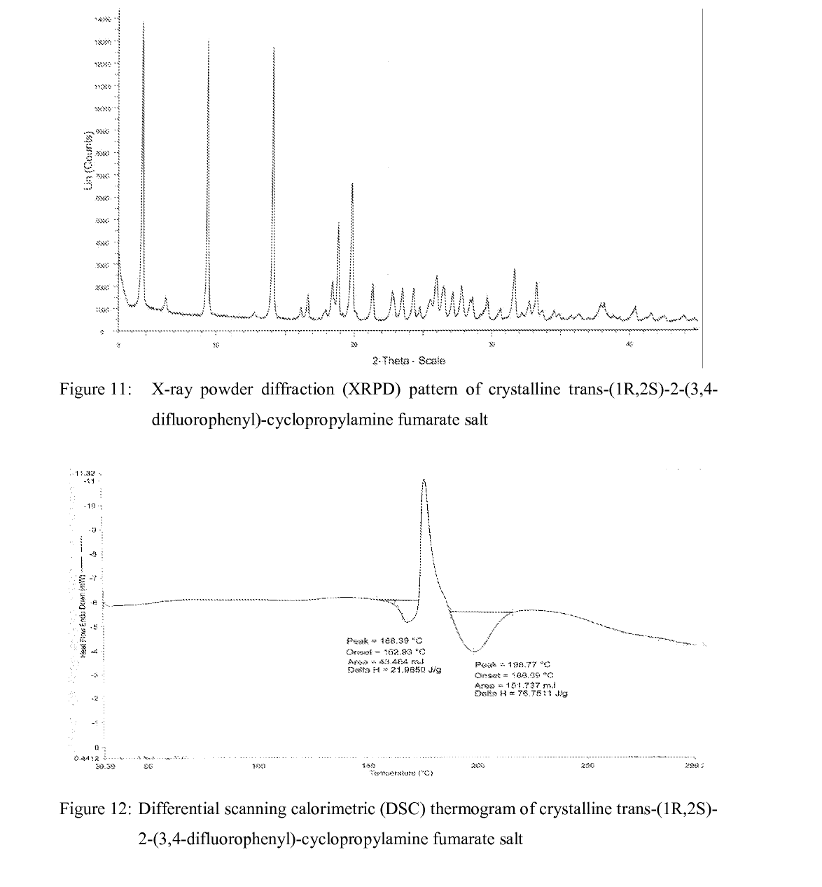

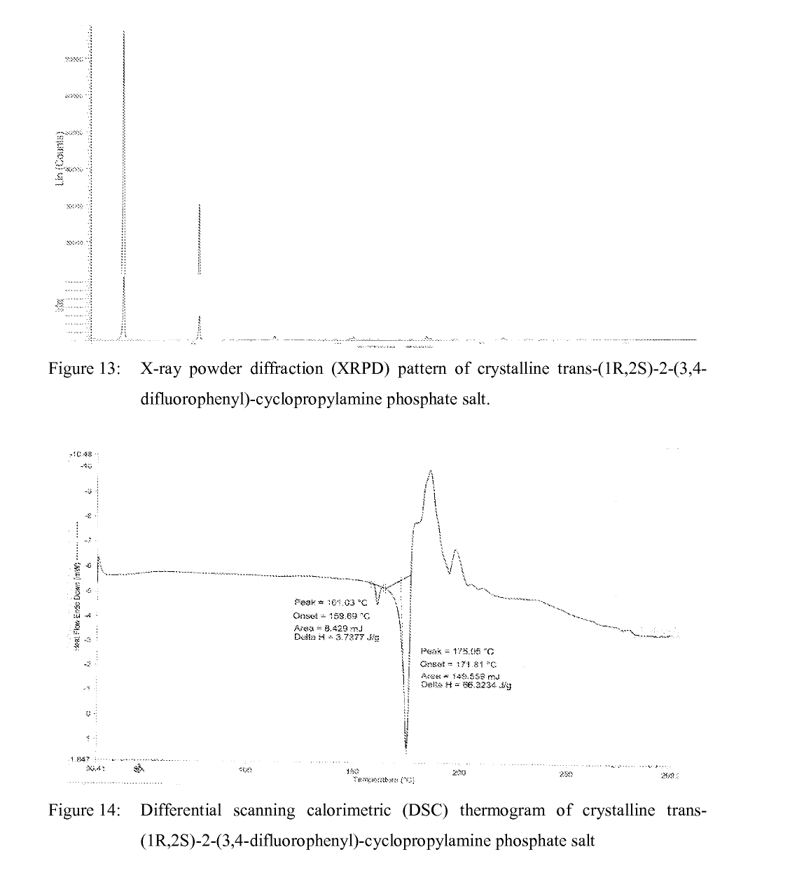

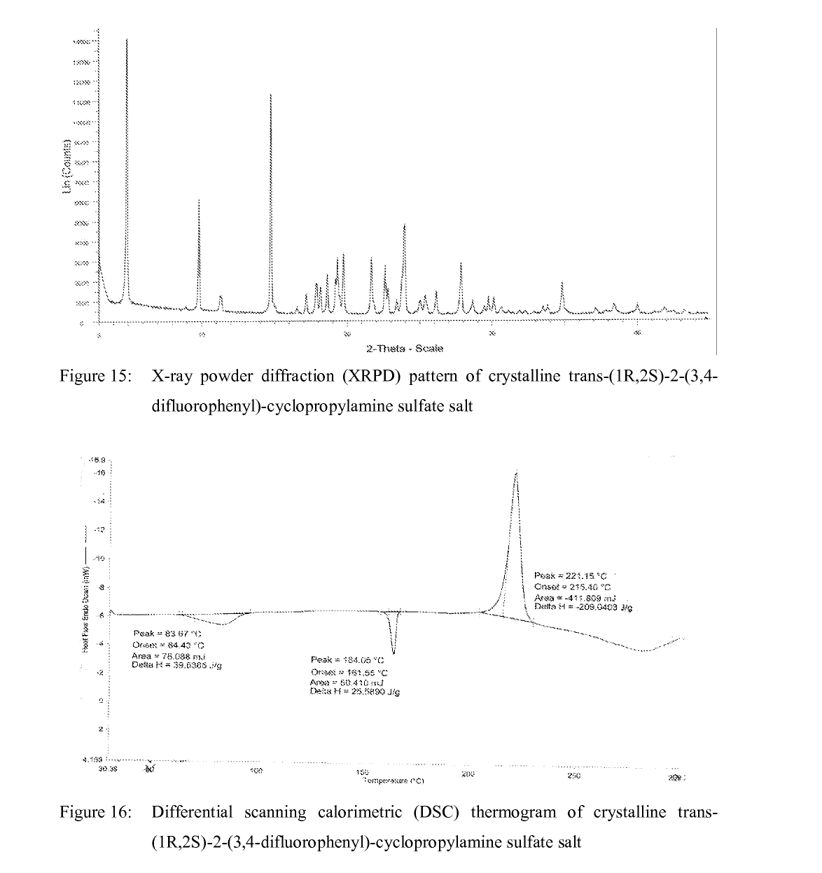

In one embodiment, the solid state forms of trans-(1R,2S)-2-(3,4-difluorophenyl)-cyclopropylamine acid addition salts have the following characteristics, wherein:
1) the solid state form of trans-(1R,2S)-2-(3,4-difluorophenyl)-cyclopropylamine tartrate salt is characterized by one or more of the following properties:
   i) a powder X-ray diffraction pattern substantially in accordance with FIG. 1;
   ii) a powder X-ray diffraction pattern having peaks at about 5.14, 6.81, 10.32, 11.96, 12.63, 14.45, 15.34, 15.54, 15.90, 16.24, 17.50, 19.67, 20.37, 20.73 and 22.46±0.2 degrees 2-theta; and
   iii) a DSC thermogram substantially in accordance with FIG. 2;
2) the solid state form of trans-(1R,2S)-2-(3,4-difluorophenyl)-cyclopropylamine di-p-toluoyl-tartrate salt is characterized by one or more of the following properties:
   i) a powder X-ray diffraction pattern substantially in accordance with FIG. 3; and
   ii) a powder X-ray diffraction pattern having peaks at about 6.79, 12.18, 12.57, 13.60, 14.37, 15.28, 18.21, 18.82, 19.26 and 23.40±0.2 degrees 2-theta;
3) the solid state form of trans-(1R,2S)-2-(3,4-difluorophenyl)-cyclopropylamine (S)-ketopinate salt is characterized by one or more of the following properties:
   i) a powder X-ray diffraction pattern substantially in accordance with FIG. 4;
   ii) a powder X-ray diffraction pattern having peaks at about 6.72, 9.49, 12.88, 13.51, 13.73, 14.37, 17.40, 17.84, 18.25, 19.14, 19.28, 19.55, 25.59, 26.23 and 27.54±0.2 degrees 2-theta; and
   iii) a DSC thermogram substantially in accordance with FIG. 5;
4) the solid state form of trans-(1R,2S)-2-(3,4-difluorophenyl)-cyclopropylamine (D)-malate salt is characterized by one or more of the following properties:
   i) a powder X-ray diffraction pattern substantially in accordance with FIG. 6;
   ii) a powder X-ray diffraction pattern having peaks at about 5.34, 10.73, 12.79, 15.11, 16.15, 17.86, 18.78, 20.07, 21.61, 22.16, 22.30, 24.08, 27.12 and 27.46±0.2 degrees 2-theta; and
   iii) a DSC thermogram substantially in accordance with FIG. 7;
5) the solid state form of trans-(1R,2S)-2-(3,4-difluorophenyl)-cyclopropylamine (D)-camphorsulfonate salt is characterized by one or more of the following properties:
   i) a powder X-ray diffraction pattern substantially in accordance with FIG. 8;
   ii) a powder X-ray diffraction pattern having peaks at about 6.73, 8.57, 13.89, 15.34, 16.66, 19.06, 19.62, 20.94, 24.66 and 26.70±0.2 degrees 2-theta; and
   iii) a DSC thermogram substantially in accordance with FIG. 9;
6) the solid state form of trans-(1R,2S)-2-(3,4-difluorophenyl)-cyclopropylamine (R)-(−)-α-methoxyphenylacetate salt is characterized by one or more of the following properties:
   i) a powder X-ray diffraction pattern substantially in accordance with FIG. 10; and
   ii) a powder X-ray diffraction pattern having peaks at about 4.85, 6.63, 7.87, 9.59, 11.57, 12.43, 12.66, 15.84, 16.36, 17.53, 17.97, 18.25, 18.77, 20.11, 20.73, 21.22, 22.42, 23.09, 23.42, 25.47 and 26.94±0.2 degrees 2-theta;
7) the solid state form of trans-(1R,2S)-2-(3,4-difluorophenyl)-cyclopropylamine fumarate salt is characterized by one or more of the following properties:
   i) a powder X-ray diffraction pattern substantially in accordance with FIG. 11;
   ii) a powder X-ray diffraction pattern having peaks at about 4.68, 9.38, 14.09, 16.61, 18.39, 18.83, 19.82, 21.33, 22.77, 23.48, 24.30, 25.96, 26.49, 27.80 and 31.65±0.2 degrees 2-theta; and
   iii) a DSC thermogram substantially in accordance with FIG. 12;
8) the solid state form of trans-(1R,2S)-2-(3,4-difluorophenyl)-cyclopropylamine phosphate salt is characterized by one or more of the following properties:
   i) a powder X-ray diffraction pattern substantially in accordance with FIG. 13;
   ii) a powder X-ray diffraction pattern having peaks at about 5.19, 10.39, 15.61, 21.08 and 26.17±0.2 degrees 2-theta; and
   iii) a DSC thermogram substantially in accordance with FIG. 14;
9) the solid state form of trans-(1R,2S)-2-(3,4-difluorophenyl)-cyclopropylamine sulfate salt is characterized by one or more of the following properties:
   i) a powder X-ray diffraction pattern substantially in accordance with FIG. 15;
   ii) a powder X-ray diffraction pattern having peaks at about 4.87, 9.78, 14.72, 17.85, 18.14, 18.61, 19.31, 19.73, 21.66, 22.61, 23.93, 27.86 and 34.85±0.2 degrees 2-theta; and
   iii) a DSC thermogram substantially in accordance with FIG. 16.

According to another aspect, there is provided a process for the preparation of an acid addition salt of trans-(1R,2S)-2-(3,4-difluorophenyl)-cyclopropylamine, wherein the acid addition salt is a tartrate salt, a di-p-toluoyl-tartrate salt, an (S)-ketopinate salt, a (D)-malate salt, a (D)-camphorsulfonate salt, a (R)-(−)-α-methoxyphenyl acetate salt, a fumarate salt, a phosphate salt or a sulfate salt, comprising:
a) providing a first solution or suspension of trans-(1R,2S)-2-(3,4-difluorophenyl)-cyclopropylamine free base in an alcoholic solvent;
b) combining the first solution or suspension with an acid to produce a second solution or suspension containing trans-(1R,2S)-2-(3,4-difluorophenyl)-cyclopropylamine acid addition salt, wherein the acid is selected from the group consisting of tartaric acid, di-p-toluoyl-tartric acid, (S)-ketopinic acid, (D)-malic acid, (D)-camphorsulfonic acid, (R)-(−)-α-methoxyphenyl acetic acid, fumaric acid, phosphoric acid and sulfuric acid; and
c) optionally, substantially removing the solvent from the second solution or suspension to obtain a residue, followed by dissolving or suspending the residue in a second solvent to produce a third solution or suspension;
d) isolating and/or recovering the solid state form of trans-(1R,2S)-2-(3,4-difluorophenyl)-cyclopropylamine acid addition salt either from the second solution or suspension obtained in step-(b) or from the third solution or suspension obtained in step-(c).

The solid state form of trans-(1R,2S)-2-(3,4-difluorophenyl)-cyclopropylamine acid addition salt obtained by the process disclosed herein is further optionally converted into highly pure trans-(1R,2S)-2-(3,4-difluorophenyl)-cyclopropylamine free base by treating with a base in a suitable solvent, or it can be used directly in the preparation of ticagrelor or a pharmaceutically acceptable salt thereof.

The process can produce solid state forms of trans-(1R,2S)-2-(3,4-difluorophenyl)-cyclopropylamine acid addition salt in substantially pure form.

The term “substantially pure solid state form of trans-(1R,2S)-2-(3,4-difluorophenyl)-cyclopropylamine acid addition salt” refers to the solid state form of trans-(1R,2S)-2-(3,4-difluorophenyl)-cyclopropylamine acid addition salt having a purity of greater than about 98 wt %, specifically greater than about 99 wt %, more specifically greater than about 99.5 wt %, and still more specifically greater than about 99.9 wt %. The purity is preferably measured by High Performance Liquid Chromatography (HPLC). For example, the purity of the solid state form of trans-(1R,2S)-2-(3,4-difluorophenyl)-cyclopropylamine acid addition salt obtained by the process disclosed herein can be about 98% to about 99.95%, or about 99% to about 99.99%, as measured by HPLC.

In one embodiment, the alcohol solvent used in step-(a) is selected from the group consisting of methanol, ethanol, n-propanol, isopropyl alcohol, isobutanol, n-butanol, tert-butanol, amyl alcohol, isoamyl alcohol, and mixtures thereof.

Specifically, the alcohol solvent is selected from the group consisting of methanol, ethanol, isopropyl alcohol, and mixtures thereof; and a more specific alcohol solvent is ethanol.

Step-(a) of providing a first solution of trans-(1R,2S)-2-(3,4-difluorophenyl)-cyclopropylamine free base includes dissolving trans-(1R,2S)-2-(3,4-difluorophenyl)-cyclopropylamine free base in the alcohol solvent, or obtaining an existing solution from a previous processing step.

In one embodiment, the trans-(1R,2S)-2-(3,4-difluorophenyl)-cyclopropylamine is dissolved in the alcohol solvent at a temperature of about 0° C. to the reflux temperature of the solvent used, specifically at about 10° C. to about 110° C., and more specifically at about 20° C. to about 50° C.

As used herein, “reflux temperature” means the temperature at which the solvent or solvent system refluxes or boils at atmospheric pressure.

In another embodiment, step-(a) of providing a suspension of trans-(1R,2S)-2-(3,4-difluorophenyl)-cyclopropylamine free base includes suspending trans-(1R,2S)-2-(3,4-difluorophenyl)-cyclopropylamine free base in the alcohol solvent while stirring at a temperature of about 0° C. to the reflux temperature of the solvent used. In one embodiment, the suspension is stirred at a temperature of about 10° C. to about 110° C. for at least 30 minutes and more specifically at a temperature of about 20° C. to about 60° C. for about 10 minutes to about 10 hours.

The first solution or suspension obtained in step-(a) is optionally stirred at a temperature of about 5° C. to the reflux temperature of the solvent used for at least 15 minutes, and specifically at a temperature of about 20° C. to the reflux temperature of the solvent used for about 20 minutes to about 8 hours.

The acid in step-(b) may be used directly or in the form of a solution containing the acid and a suitable solvent. The suitable solvent used for dissolving the acid is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Combining of the first solution or suspension with acid in step-(b) is done in a suitable order, for example, the first solution or suspension is added to the acid, or alternatively, the acid is added to the first solution or suspension. The addition is, for example, carried out drop wise or in one portion or in more than one portion. The addition is specifically carried out at a temperature of about 0° C. to the reflux temperature of the solvent used, more specifically at about 10° C. to about 110° C., and most specifically at about 20° C. to about 60° C. under stirring. After completion of addition process, the resulting solution is stirred at a temperature of about 0° C. to the reflux temperature of the solvent used for at least 10 minutes, specifically at about 10° C. to about 110° C. for about 20 minutes to about 25 hours, and more specifically at a temperature of about 20° C. to about 60° C. for about 30 minutes to about 8 hours to produce a second solution or suspension.

The second solution obtained in step-(b) is optionally subjected to carbon treatment or silica gel treatment. The carbon treatment or silica gel treatment is carried out by methods known in the art, for example, by stirring the solution with finely powdered carbon or silica gel at a temperature of below about 80° C. for at least 15 minutes, specifically at a temperature of about 40° C. to about 70° C. for at least 30 minutes; and filtering the resulting mixture through hyflo to obtain a filtrate containing trans-(1R,2S)-2-(3,4-difluorophenyl)-cyclopropylamine acid addition salt by removing charcoal or silica gel. Specifically, the finely powdered carbon is an active carbon. A specific mesh size of silica gel is 40-500 mesh, and more specifically 60-120 mesh.

The term “substantially removing” the solvent refers to at least 50%, specifically greater than about 80%, more specifically greater than about 90%, still more specifically greater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent under inert atmosphere, or a combination thereof, to substantial elimination of total solvent present in the reaction mass.

In one embodiment, the distillation process can be performed at atmospheric pressure or reduced pressure. Specifically, the distillation is carried out at a temperature of about 30° C. to about 110° C., more specifically at about 40° C. to about 90° C., and most specifically at about 45° C. to about 80° C.

Specifically, the solvent is removed at a pressure of about 760 mm Hg or less, more specifically at about 400 mm Hg or less, still more specifically at about 80 mm Hg or less, and most specifically from about 30 to about 80 mm Hg.

The residue containing trans-(1R,2S)-2-(3,4-difluorophenyl)-cyclopropylamine acid addition salt obtained in step-(c) is dissolved or suspended in the second solvent a temperature of about 0° C. to the reflux temperature of the solvent used, specifically at about 20° C. to about 110° C., and more specifically at about 25° C. to about 80° C. In one embodiment, the solution or suspension is stirred at a temperature of about 20° C. to about 110° C. for at least 10 minutes and more specifically at a temperature of about 25° C. to about 80° C. for about 20 minutes to about 10 hours.

Exemplary second solvent used in step-(c) includes, but is not limited to, water, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the second solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Specifically, the second solvent is selected from the group consisting of tetrahydrofuran, dioxane, diethyl ether, diisopropyl ether, monoglyme, diglyme, and mixtures thereof; and more specifically diethyl ether and diisopropyl ether.

The isolation of pure solid state form of trans-(1R,2S)-2-(3,4-difluorophenyl)-cyclopropylamine acid addition salt in step-(d) is carried out by forcible crystallization, spontaneous crystallization, substantial removal of the solvent from the solution or suspension, or a combination thereof.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

The term “Anti-solvent” refers to a solvent which when added to an existing solution of a substance reduces the solubility of the substance.

In one embodiment, the crystallization is carried out by cooling the solution under stirring at a temperature of below 30° C. for at least 10 minutes, specifically at about 0° C. to about 30° C. for about 30 minutes to about 20 hours.

The recovering in step-(d) is carried out by methods such as filtration, filtration under vacuum, decantation, centrifugation, or a combination thereof. In one embodiment, solid state form of trans-(1R,2S)-2-(3,4-difluorophenyl)-cyclopropylamine salt is recovered by filtration employing a filtration media of, for example, a silica gel or celite.

Ticagrelor or a pharmaceutically acceptable salt thereof can be prepared in high purity by using the solid state forms of trans-(1R,2S)-2-(3,4-difluorophenyl)-cyclopropylamine acid addition salts disclosed herein, by the methods known in the art.

### Instrumental Details:

### X-Ray Powder Diffraction (P-XRD):

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45° 2-theta; step width=0.01579°; and measuring time per step=0.11 second.

### Differential Scanning Calorimetry (DSC):

DSC (Differential Scanning calorimetry) measurements were performed with a Differential Scanning calorimeter (Diamond DSC, Perkin-Elmer) at a scan rate of 10° C. per minute. The nitrogen gas purge was at 40 ml/min. The instrument was calibrated for temperature and heat flow using indium as standards. The samples were encapsulated in to closed aluminium pans without hole subsequently crimped to ensure a tight seal. Data acquisition and analysis were performed using pyris software.

The following examples are given for the purpose of illustrating the present disclosure and should not be considered as limitation on the scope or spirit of the disclosure.

### EXAMPLES

### Example 1

### Preparation of 3,4-Difluorostyrene

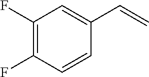

Methyltriphenylphosphonium bromide (71 g, 0.2111 mol), 1,8-diazabicyclo[5.4.0]undec-7-ene (35.37 g, 0.2311 mol) and toluene (75 ml) were taken into a clean and dry reaction assembly. The resulting mixture was heated at 40-45° C., followed by stirring for 30 minutes. 3,4-Difluorobenzaldehyde (15 g, 0.1055 mol) was slowly added to the above hot solution and the reaction mixture was heated at reflux temperature, followed by maintaining for 6 hours at reflux. After completion of the reaction, the mass was cooled to 25-30° C., followed by washing with water (2×250 ml). The resulting mass was distilled under reduced pressure while maintaining the temperature at below 50° C. to give 3,4-difluorostyrene.

### Example 2

### Preparation of Ethyldiazoacetate Solution in Toluene

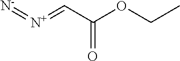

Sodium nitrite (13 g, 0.188 mol) was added to a stirred solution of sodium tetraborate decahydrate (2.48 g, 0.0065 mol) in water (50 ml) at 25° C., followed by the addition of glycine ethyl ester hydrochloride salt (25 g, 0.179 mol). Upon complete dissolution, toluene (60 ml) was added to the mass, and the resulting biphasic mixture was cooled to 0° C. A 2% (w/w) solution of phosphoric acid in water was added to the resulting mass over a period of 30 minutes while maintaining the temperature at below 20° C. until the pH was adjusted between 3.7 and 4.5 (addition of 90 ml resulted in a pH of 3.95). The layers were separated, followed by washing of the organic layer successively with water (25 ml) and 8% (w/w) of aqueous sodium bicarbonate solution (2×50 ml). The combined aqueous washes were neutralized with a 20 wt % solution of phosphoric acid in water and the washes were discarded. The organic layer was held overnight at 10° C. before being used in next step.

### Example 3

### Preparation of Ethyl (1R,2R)-trans-2-(3,4-difluorophenyl)-1-cyclopropanecarboxylate

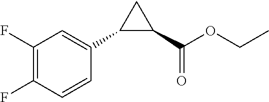 [SMILES: CCOC(=O)[C@@H]1C[C@H]1c1ccc(F)c(F)c1]

The solution of 3,4-difluorostyrene in toluene (obtained in example 1) was take into a clean and dry reaction assembly, followed by the addition of dichloro(p-cymene)ruthenium(II) dimer (1 g) and (S,S)-2,6-bis(4-isopropyl-2-oxazolin-2-yl)pyridine (1 g) under stirring. The resulting solution was heated at 50-55° C., followed by the addition of ethyl diazoacetate solution in toluene (obtained in example 2) over a period of 8 to 10 hours while maintaining the temperature between 50-55° C. After completion of the addition process, the reaction mass was further stirred for 1 hour at 50-55° C., followed by cooling to 25-30° C. Water (100 ml) was added to the cooled reaction mass, followed by stirring for 5 minutes. The layers were separated and the aqueous layer was extracted with toluene (100 ml). The both toluene layers were combined, followed by washing of the combined toluene layer with water (100 ml) and 50% acetic acid solution (100 ml) in water (100 ml). The toluene layer was evaporated under reduced pressure to obtain the crude ethyl (1R,2R)-trans-2-(3,4-difluorophenyl)-1-cyclopropanecarboxylate as an oil (19.5 g) which was directly used in next step.

### Example 4

### Preparation of (1R,2R)-trans-2-(3,4-difluorophenyl)-1-cyclopropanecarboxylic acid

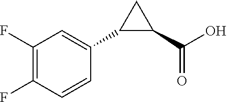

A solution of crude ethyl (1R,2R)-trans-2-(3,4-difluorophenyl)-1-cyclopropane carboxylate (19.5 g, obtained in example 3) in methanol (130 ml) and 30% aqueous solution of sodium hydroxide (20.85 g) were taken into a clean reaction assembly. The mixture was heated at 60-65° C. and maintained while stirring for 2 hours. The resulting mixture was concentrated under reduced pressure, followed by the addition of toluene (100 ml) and water (50 ml). The mixture was acidified with concentrated hydrochloric acid to adjust the pH less than 1.5. The organic layer was separated and the aqueous layer was extracted with toluene (100 ml). Both the toluene layers were combined and washed with water (100 ml). The organic layer was dried over sodium sulfate and concentrated under reduced pressure to obtain (1R,2R)-trans-2-(3,4-difluorophenyl)-1-cyclopropanecarboxylic acid which was further purified by preparing the (S)-(−)-methylbenzyl amine salt in isopropyl alcohol, followed by acidification to obtain pure (1R,2R)-trans-2-(3,4-difluorophenyl)-1-cyclopropanecarboxylic acid (11 g).

¹H-NMR (CDCl₃,δ): 1.33 (1H, m), 1.64 (1H, m), 1.82 (1H, m), 2.55 (1H, m), 6.82 (2H, m), 7.03 (1H, m), 10.0 (1H, broad); Mass [M-H]: 196.60.

### Example 5

### Preparation of 3,4-Difluorostyrene

Methyltriphenylphosphonium bromide (251.31 g, 0.7037 mol), 1,8-diazabicyclo[5.4.0]undec-7-ene (117.84 g, 0.7741 mol) and toluene (250 ml) were taken into a clean and dry reaction assembly. The resulting mixture was heated at 40-45° C., followed by stirring for 30 minutes. 3,4-Difluorobenzaldehyde (50 g, 0.3518 mol) was slowly added to the hot solution and the reaction mixture was heated at reflux temperature, followed by maintaining for 5 hours at reflux. After completion of the reaction, the mass was cooled to 25-30° C., followed by washing with water (2×250 ml). The resulting mass was distilled under reduced pressure while maintaining the temperature at below 50° C. to give 3,4-difluorostyrene.

### Example 6

### Preparation of Ethyldiazoacetate Solution in Toluene

Sodium nitrite (25.82 g, 0.3742 mol) was added to a stirred solution of sodium tetraborate decahydrate (4.876 g, 0.0127 mol) in water (100 ml) at 25° C., followed by the addition of glycine ethyl ester hydrochloride salt (50 g, 0.3581 mol). Upon complete dissolution, toluene (116 ml) was added to the mass, and the resulting biphasic mixture was cooled to 0° C. 2% (w/w) solution of phosphoric acid in water was added to the resulting mass over a period of 30 minutes while maintaining the temperature at below 20° C. until the pH was adjusted between 3.7 and 4.5 (addition of 140 ml resulted in a pH of 3.99). The layers were separated, followed by washing of the organic layer successively with water (50 ml) and 8% (w/w) of aqueous sodium bicarbonate solution (2×100 ml). The combined aqueous washes were neutralized with a 20 wt % solution of phosphoric acid in water and the washes were discarded. The organic layer was used in the next step for cyclopropanation.

### Example 7

### Preparation of Ethyl (1R,2R)-trans-2-(3,4-difluorophenyl)-1-cyclopropanecarboxylate

The solution of 3,4-difluorostyrene in toluene (obtained in example 5) was take into a clean and dry reaction assembly, followed by the addition of dichloro(p-cymene)ruthenium(II) dimer (2.5 g) and (S,S)-2,6-bis(4-isopropyl-2-oxazolin-2-yl)pyridine (2.5 g) under stirring. The resulting solution was heated at 50-55° C., followed by the addition of ethyl diazoacetate solution in toluene (obtained in example 6) over a period of 8 to 10 hours while maintaining the temperature between 50-55° C. Upon complete addition, the reaction mass was further stirred for 10 hours at 50-55° C., followed by cooling to 25-30° C. Water (200 ml) was added to the cooled reaction mass, followed by stirring for 5 minutes. The layers were separated and the aqueous layer was extracted with toluene (200 ml). Both toluene layers were combined, followed by washing successively with water (300 ml), 50% acetic acid solution (300 ml) in water (300 ml). The toluene layer was evaporated under reduced pressure to obtain the crude ethyl (1R,2R)-trans-2-(3,4-difluorophenyl)-1-cyclopropanecarboxylate as an oil (50 g), which was directly used in next step.

### Example 8

### Preparation of (1R,2R)-trans-2-(3,4-difluorophenyl)-1-cyclopropanecarboxylic acid

A solution of crude ethyl (1R,2R)-trans-2-(3,4-difluorophenyl)-1-cyclopropane carboxylate (40 g, obtained in example 7) in methanol (267 ml) and a 30% aqueous solution of sodium hydroxide (42.77 g) were taken into a clean reaction assembly. The mixture was heated at 60-65° C. and maintained while stirring for 2 hours. The resulting mixture was concentrated under reduced pressure, followed by the addition of toluene (200 ml) and water (100 ml). The mixture was acidified with concentrated hydrochloric acid to adjust the pH less than 1.5. The organic layer was separated and the aqueous layer was extracted with toluene (200 ml). Both the toluene layers were combined and washed with water (200 ml). The organic layer was dried over sodium sulfate and concentrated under reduced pressure. The residue obtained was dissolved in isopropyl alcohol (200 ml), followed by the addition of (S)-(−)-α-methylbenzylamine (10.5 g). The resulting slurry was stirred overnight, followed by filtration. The wet amine salt was dried under reduced pressure, the dried salt was suspended in water (100 ml), followed by acidification to adjust the pH to below 2 by adding concentrated hydrochloric acid. The resulting acidic solution was extracted with toluene (100 ml), followed washing of the toluene layer with water (100 ml). The toluene layer was dried over sodium sulfate and then concentrated under reduced pressure to produce pure (1R,2R)-trans-2-(3,4-difluorophenyl)-1-cyclopropanecarboxylic acid (10 g).

¹H-NMR (CDCl₃,δ): 1.33 (1H, m), 1.65 (1H, m), 1.83 (1H, m), 2.56 (1H, m), 6.83 (2H, m), 7.04 (1H, m); [R]²⁰_{D}=−257.6° (c1, CHCl₃).

### Example 9

### Preparation of trans-(1R,2S)-2-(3,4-difluorophenyl)-cyclopropylamine

(1R,2R)-trans-2-(3,4-difluorophenyl)-1-cyclopropanecarboxylic acid (5 g, 0.0252 mol, obtained in example 4 or 8) and diphenylphosphoryl azide (7.64 g, 0.277 mol) were dissolved in toluene (50 ml), triethylamine (5.1 g, 0.0505 mol) was added to the solution, followed by stirring under heating in an oil bath (at 125° C.) for 1 hour. The reaction mixture was concentrated under reduced pressure to yield an isocyanate compound. The isocyanate compound was dissolved in 1,4-dioxane (44 ml), followed by the addition of water (22 ml) and concentrated hydrochloric acid (22 ml) and then stirring under heating in an oil bath (at 50° C.) for 2 hours. Subsequently, water (50 ml) was added to the reaction mixture, and the mixture was washed with toluene (2×50 ml). The pH of the resulting aqueous layer was adjusted to 10 to 11 using 30% aqueous sodium hydroxide solution under ice-cooling, followed by extraction with toluene (2×50 ml). The organic layer was washed with saturated brine (50 ml), dried over sodium sulfate anhydrous, and then filtered. The filtrate was concentrated under reduced pressure to yield 2.5 g of pure trans-(1R,2S)-2-(3,4-difluorophenyl)-cyclopropylamine as a pale greenish yellow oil.

¹H-NMR (CDCl₃,δ): 0.88 (1H, m), 1.05 (1H, m), 1.70 (2H, bs), 1.83 (1H, m), 2.49 (1H, m), 6.72 (2H, m), 6.97 (1H, m); and [R]²⁰_{D}=−91.6° (c1, CHCl₃).

### Example 10

### Preparation of trans-(1R,2R)-2-(3,4-difluorophenyl)-N-hydroxycyclopropanecarboxamide

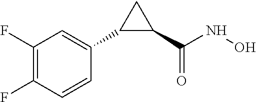

A mixture of (1R,2R)-trans-2-(3,4-difluorophenyl)-1-cyclopropanecarboxylic acid (2 g, 0.0101 mol) and tetrahydrofuran (16 ml) was cooled to 0-5° C., followed by the addition of triethyl amine (1.083 g, 0.107 mol). A solution of isobutyl chloroformate (1.448 g, 0.106 ml) in tetrahydrofuran (4 ml) was slowly added to the resulting mixture while maintaining the temperature at about 0-5° C., followed by stirring for 1 hour. To the mixture was added 50% aqueous hydroxylamine solution prepared by neutralizing 50% aqueous hydroxylamine hydrochloride (9.05 g) by triethyl amine (20 ml), followed by stirring for 20 minutes at 5-10° C. Subsequently, water (10 ml) was added to the reaction mixture, and the mixture was extracted with ethyl acetate (50 ml and 20 ml). The organic layer was washed with saturated brine (20 ml), dried over sodium sulfate anhydrous, and then filtered. The filtrate was concentrated under reduced pressure to yield 2.14 g of trans-(1R,2R)-2-(3,4-difluorophenyl)-N-hydroxycyclopropanecarboxamide.

Mass [M-H]: 212.0

### Example 11

### Preparation of trans-(1R,2R)—N-(acetyloxy)-2-(3,4-difluorophenyl)cyclopropane carboxamide

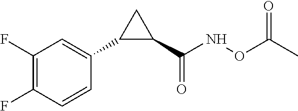

A mixture of trans-(1R,2R)-2-(3,4-difluorophenyl)-N-hydroxycyclopropanecarboxamide (2.1 g, 0.00985 mol) and tetrahydrofuran (10 ml) was mixed with pyridine (1.043 g, 0.0132 mol), followed by slow addition of acetic anhydride (1.066 g, 0.0104 mol) while maintaining the temperature at about 25-30° C. and then stirring for 20 minutes at the same temperature. After completion of the reaction, ethyl acetate (50 ml) and 1N hydrochloric acid (10 ml) were added, followed by layer separation. Aqueous layer was extracted with ethyl acetate (50 ml). The organic layer was washed with water saturated brine (10 ml), aqueous sodium bicarbonate solution (10 ml), followed by drying over sodium sulfate anhydrous, and then filtering. The filtrate was concentrated under reduced pressure to yield 2 g of trans-(1R,2R)—N-(acetyloxy)-2-(3,4-difluorophenyl)cyclopropane carboxamide.

Mass [M-H]: 254.1

### Example 12

### Preparation of trans-(1R,2S)-2-(3,4-difluorophenyl)-cyclopropylamine

A mixture of trans-(1R,2R)—N-(acetyloxy)-2-(3,4-difluorophenyl)cyclopropane carboxamide (1.8 g) and tetrahydrofuran (21 ml) was heated to 40-45° C., followed by the addition of water (1.91 ml). The temperature of the reaction mass was increased to 50-55° C., followed by the addition of 1,8-diazabicyclo[5.4.0]undecane-7-ene (DBU) (1.43 g). The resulting mixture was heated to reflux temperature and then maintained for 5 hours. After completion of the reaction, the reaction mass was cooled to 25-30° C., followed by the addition of isopropyl acetate (50 ml) and saturated ammonium chloride solution (20 ml). The resulting layers were separated, followed by washing the organic layer with saturated ammonium chloride (20 ml), water (20 ml). The resulting organic layer was dried over sodium sulfate anhydrous and then filtered. The filtrate was concentrated under reduced pressure to yield 1.4 g of trans-(1R,2S)-2-(3,4-difluorophenyl)-cyclopropylamine.

### Example 13

### Preparation of trans-(1R,2S)-2-(3,4-difluorophenyl)-cyclopropylamine tartrate salt

Trans-(1R,2S)-2-(3,4-difluorophenyl)-cyclopropylamine (2 g) was dissolved in ethanol (5 ml) at 25-30° C., followed by slow addition of a solution of L-tartaric acid (1.78 g) in ethanol (25 ml) at 20-25° C. The slurry was stirred further 30 minutes at 20-25° C. The precipitated product was collected by filtration, washed with ethanol (5 ml) and then dried to give 2.9 g of trans-(1R,2S)-2-(3,4-difluorophenyl)-cyclopropylamine tartrate salt.

### Example 14

### Preparation of trans-(1R,2S)-2-(3,4-difluorophenyl)-cyclopropylamine di-p-toluoyl tartrate salt

Trans-(1R,2S)-2-(3,4-difluorophenyl)-cyclopropylamine (2 g) was dissolved in ethanol (5 ml) at 25-30° C., followed by slow addition of a solution of di-p-toluoyl-L-tartaric acid (4.5) in ethanol (25 ml) at 25-30° C. The slurry was stirred for 1 hour at 25-30° C. The precipitated product was collected by filtration, washed with ethanol (5 ml) and then dried to give 5.5 g of trans-(1R,2S)-2-(3,4-difluorophenyl)-cyclopropylamine di-p-toluoyl tartrate salt.

### Example 15

### Preparation of trans-(1R,2S)-2-(3,4-difluorophenyl)-cyclopropylamine (S)-ketopinate salt

Trans-(1R,2S)-2-(3,4-difluorophenyl)-cyclopropylamine (0.88 g) was dissolved in ethanol (3 ml) at 25-30° C., followed by slow addition of a solution of (S)-(+)-ketopinic acid (0.95 g) in ethanol (7 ml) at 25-30° C. The slurry was stirred for 30 minutes at 25-30° C. The precipitated product was collected by filtration and then dried to give 0.5 g of trans-(1R,2S)-2-(3,4-difluorophenyl)-cyclopropylamine (S)-ketopinate salt.

### Example 16

### Preparation of trans-(1R,2S)-2-(3,4-difluorophenyl)-cyclopropylamine (D)-malate salt

Trans-(1R,2S)-2-(3,4-difluorophenyl)-cyclopropylamine (2 g) was dissolved in ethanol (5 ml) at 25-30° C., followed by slow addition of a solution of (D)-(+)-malic acid (1.58 g) in ethanol (15 ml) at 25-30° C. The slurry was stirred for 30 minutes at 25-30° C. The precipitated product was collected by filtration and then dried to give 2.46 g of trans-(1R,2S)-2-(3,4-difluorophenyl)-cyclopropylamine (D)-malate salt.

### Example 17

### Preparation of trans-(1R,2S)-2-(3,4-difluorophenyl)-cyclopropylamine (D)-camphor sulfonate salt

Trans-(1R,2S)-2-(3,4-difluorophenyl)-cyclopropylamine (2 g) was dissolved in ethanol (5 ml) at 25-30° C., followed by slow addition of a solution of (D)-(+)-camphorsulphonic acid (3.0 g) in ethanol (15 ml) at 25-30° C. The slurry was stirred for 1 hour at 25-30° C. The solvent was evaporated under reduced pressure to give 4 g of trans-(1R,2S)-2-(3,4-difluorophenyl)-cyclopropylamine (D)-camphor sulfonate salt.

### Example 18

### Preparation of trans-(1R,2S)-2-(3,4-difluorophenyl)-cyclopropylamine fumarate

Trans-(1R,2S)-2-(3,4-difluorophenyl)-cyclopropylamine (1 g) was dissolved in ethanol (10 ml) at 25-30° C., followed by the addition of fumaric acid (0.7 g) at 25-30° C. The slurry was stirred for 30 minutes at 25-30° C. The precipitated product was collected by filtration, washed with ethanol (2×5 ml) and then dried to give 0.9 g of trans-(1R,2S)-2-(3,4-difluorophenyl)-cyclopropylamine fumarate.

### Example 19

### Preparation of trans-(1R,2S)-2-(3,4-difluorophenyl)-cyclopropylamine phosphate

Trans-(1R,2S)-2-(3,4-difluorophenyl)-cyclopropylamine (1 g) was dissolved in ethanol (10 ml) at 25-30° C., followed by the addition of o-phosphoric acid (0.6 g) at 25-30° C. The slurry was stirred for 30 minutes at 25-30° C. The precipitated product was collected by filtration, washed with ethanol (2×5 ml) and then dried to give 1.1 g of trans-(1R,2S)-2-(3,4-difluorophenyl)-cyclopropylamine phosphate.

### Example 20

### Preparation of trans-(1R,2S)-2-(3,4-difluorophenyl)-cyclopropylamine sulfate

Trans-(1R,2S)-2-(3,4-difluorophenyl)-cyclopropylamine (1 g) was dissolved in ethanol (10 ml) at 25-30° C., followed by the addition of sulfuric acid (0.6 g) at 25-30° C. The slurry was stirred for 30 minutes at 25-30° C. The precipitated product was collected by filtration, washed with ethanol (2×5 ml) and then dried to give 0.9 g of trans-(1R,2S)-2-(3,4-difluorophenyl)-cyclopropylamine sulfate.

### Example 21

### Preparation of trans-(1R,2S)-2-(3,4-difluorophenyl)-cyclopropylamine (R)-(−)-α-methoxyphenyl acetate

Trans-(1R,2S)-2-(3,4-difluorophenyl)-cyclopropylamine (0.41 g) was dissolved in methanol (3 ml) at 25-30° C., followed by slow addition of a solution of (R)-(−)-α-methoxyphenyl acetic acid (0.403 g) in methanol (5 ml) at 20-25° C. The slurry was stirred further 30 minutes at 20-25° C. The precipitated product was collected by filtration and then dried to give 0.22 g of trans-(1R,2S)-2-(3,4-difluorophenyl)-cyclopropylamine (R)-α-methoxyphenyl acetate salt.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

We claim:

1. A process for preparing substituted phenylcyclopropylamine derivatives of formula II: 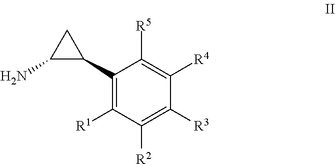
or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring in formula II is substituted with one or more halogen atoms, wherein the halogen atom is F, Cl, Br or I; comprising:
a) reacting a halogen substituted benzaldehyde compound of formula VIII: 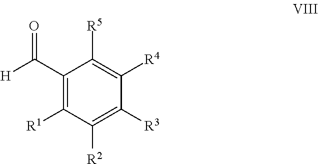
wherein R¹, R², R³, R⁴ and R⁵ are as defined in formula II; with a methyltriphenyl phosphonium halide (Wittig reagent) of formula VII: 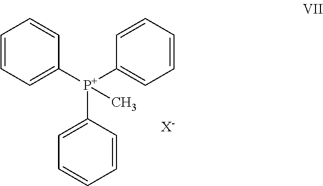
wherein ‘X’ is a halogen, selected from the group consisting of Cl, Br and I;
in the presence of a first base in a first solvent to produce a substituted styrene compound of formula VI: 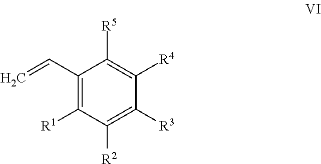
wherein R¹, R², R³, R⁴ and R⁵ are as defined in formula II;
b) reacting the compound of formula VI with a diazoester compound of formula V: 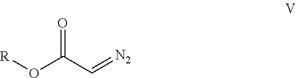
wherein ‘R’ is an alkyl, cycloalkyl, aryl or aralkyl group; in the presence of a metal catalyst and a chiral ligand in a second solvent to produce a substituted cyclopropanecarboxylate compound of formula IV: 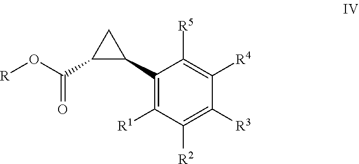
or a stereochemically isomeric form or a mixture of stereo chemically isomeric forms thereof, wherein R, R¹, R², R³, R⁴ and R⁵ are as defined in formula II;
c) hydrolyzing the ester compound of formula IV with an acid or a second base in a third solvent to produce a substituted cyclopropanecarboxylic acid compound of formula III: 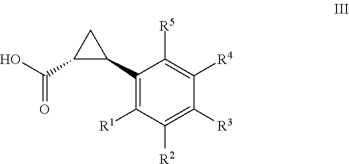
or a stereochemically isomeric form or a mixture of stereochemically isomeric forms thereof;
d) optionally, purifying the cyclopropanecarboxylic acid compound of formula III by treating with a chiral amine in a fourth solvent to produce a pure chiral amine salt of the compound of formula III;
e) optionally, acidifying the chiral amine salt of the compound of formula III with an acid to produce a pure cyclopropanecarboxylic acid compound of formula III;
f) reacting the cyclopropanecarboxylic acid compound of formula III or a chiral amine salt thereof obtained in step-(c), (d) or (e) with an azide compound, with the proviso that the azide does not include sodium azide, in the presence a third base in a fifth solvent to produce an isocyanate intermediate, followed by subjecting to acidic hydrolysis with an acid in a sixth solvent and then basifying with a fourth base to produce the substituted phenylcyclopropylamine derivatives of formula II or a stereochemically isomeric form or a mixture of stereochemically isomeric forms thereof, and optionally converting the compound of formula II obtained into an acid addition salt thereof.

2. The process of claim 1, wherein the halogen atom ‘X’ in the compound of formula VII is Cl or Br; and wherein the halogen atom in the compounds of formulae II, III, IV, VI and VIII is F.

3. The process of claim 1, wherein the halogen atom ‘X’ in the compound of formula VII is Br; and wherein the R¹, R² and R⁵ in the compounds of formulae II, III, IV, VI and VIII are H, and wherein the R³ and R⁴ are F.

4. The process of claim 1, wherein the first solvent used in step-(a) is selected from the group consisting of an ester, a nitrile, a hydrocarbon, a cyclic ether, an aliphatic ether, a polar aprotic solvent, and mixtures thereof; wherein the second solvent used in step-(b) is selected from the group consisting of a ketone, an ester, a hydrocarbon, a chlorinated hydrocarbon, a cyclic ether, an aliphatic ether, and mixtures thereof; wherein the third solvent used in step-(c) is selected from the group consisting of water, an alcohol, a ketone, a cyclic ether, an aliphatic ether, a hydrocarbon, a chlorinated hydrocarbon, a nitrile, and mixtures thereof; wherein the fourth solvent used in step-(d) is selected from the group consisting of water, an alcohol, a ketone, a cyclic ether, an aliphatic ether, a hydrocarbon, a chlorinated hydrocarbon, a nitrile, and mixtures thereof; wherein the fifth solvent used in step-(f) is selected from the group consisting of a ketone, an ester, a hydrocarbon, a chlorinated hydrocarbon, a cyclic ether, an aliphatic ether, and mixtures thereof; and wherein the sixth solvent used for hydrolysis in step-(f) is selected from the group consisting of water, a ketone, an ester, a hydrocarbon, a chlorinated hydrocarbon, a cyclic ether, an aliphatic ether, and mixtures thereof.

5. The process of claim 4, wherein the first solvent used in step-(a) is toluene; wherein the second solvent used in step-(b) is selected from the group consisting of toluene, tetrahydrofuran, 2-methyl tetrahydrofuran, and mixtures thereof; wherein the third solvent used in step-(c) is selected from the group consisting of water, methanol, ethanol, n-propanol, isopropanol, and mixtures thereof; wherein the fourth solvent used in step-(d) is selected from the group consisting of water, methanol, ethanol, n-propanol, isopropanol, and mixtures thereof; wherein the fifth solvent used in step-(f) is selected from the group consisting of toluene, tetrahydrofuran, 2-methyl tetrahydrofuran, and mixtures thereof; and wherein the sixth solvent used in step-(f) is selected from the group consisting of water, dioxane, tetrahydrofuran, 2-methyl tetrahydrofuran, and mixtures thereof.

6. The process of claim 1, wherein the Wittig reagent used in step-(a) is selected from the group consisting of methyl triphenylphosphonium chloride, methyl triphenylphosphonium bromide and methyl triphenylphosphonium iodide; wherein the diazoester compound of formula V used in step-(b) is ethyl diazoacetate, isopropyl diazoacetate, tert-butyl diazoacetate, benzyl diazoacetate, 1 or d-menthyl diazoacetate, or butylated toluene diazoacetate; wherein the metal catalyst used in step-(b) is selected from the group consisting of chlorides, bromides, acetates and fluoroalkyl acetates of a metal, wherein the metal is selected from cobalt, copper, chromium, iron, manganese, aluminium, ruthenium and rhodium; wherein the chiral ligand employed for facilitating the asymmetric cyclopropanation reaction in step-(b) is selected from the group consisting of bisoxazoline compounds, substituted salicylaldimines, salens, optically active Schiff bases, bipyridines, bisazaferrocene, dirhodium(II)carboxylates and dirhodium(II)carboxamidates; wherein the acid used in step-(c) is selected from the group consisting of methanesulfonic acid, trifluoromethanesulfonic acid, trifluoroacetic acid, hydrochloric acid, sulfuric acid, and mixtures thereof; wherein the second base used in step-(c) is selected from the group consisting of sodium hydroxide, potassium hydroxide, lithium hydroxide, calcium hydroxide, magnesium hydroxide, tetra-n-butyl ammonium hydroxide, and mixtures thereof; wherein the chiral amine used in step-(d) is (S)-(−)-methylbenzylamine; wherein the azide used in step-(f) is selected from the group consisting of diethylphosphoryl azide, diisopropylphosphoryl azide, di-tert-butylphosphoryl azide, dibutylphosphoryl azide, dibenzylphosphoryl azide, di-1 or d-menthylphosphoryl azide, and diphenylphosphoryl azide; and wherein the acid used for facilitating the hydrolysis of isocyanate intermediate in step-(f) is selected from the group consisting of methanesulfonic acid, trifluoromethanesulfonic acid, trifluoroacetic acid, hydrochloric acid and sulfuric acid.

7. The process of claim 6, wherein the Wittig reagent used in step-(a) is methyl triphenylphosphonium bromide; wherein the diazoester compound of formula V used in step-(b) is ethyl diazoacetate; wherein the metal catalyst used in step-(b) is dichloro(p-cymene)ruthenium(II) dimer; and wherein the second base used in step-(c) is sodium hydroxide.

8. The process of claim 1, wherein the stereochemically isomeric form of the substituted phenylcyclopropylamine derivative of formula II obtained in step-(f) is trans-(1R,2S)-2-(3,4-difluorophenyl)-cyclopropylamine of formula IIa (formula II, wherein R¹, R² and R⁵ are H, and R³ and R⁴ are F):

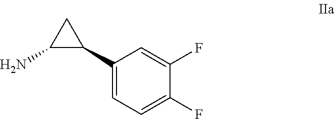 [SMILES: N[C@@H]1C[C@H]1c1ccc(F)c(F)c1]

9. The process of claim 1, wherein the stereochemically isomeric form of the substituted phenylcyclopropylamine derivative of formula II obtained in step-(f) is trans-(1S,2R)-2-(3,4-difluorophenyl)-cyclopropylamine of formula IIb (formula II, wherein R¹, R² and R⁵ are H, and R³ and R⁴ are F):

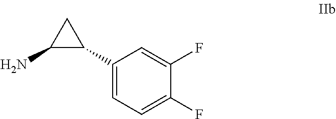 [SMILES: N[C@H]1C[C@@H]1c1ccc(F)c(F)c1]

10. A process for preparing substituted phenylcyclopropylamine derivatives of formula II: 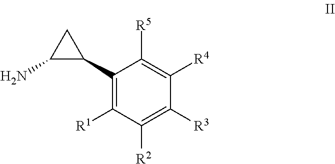
or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring in formula II is substituted with one or more halogen atoms, wherein the halogen atom is F, Cl, Br or I; comprising:
a) reacting the substituted cyclopropanecarboxylic acid compound of formula III: 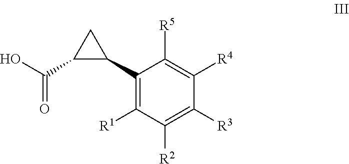
or a stereochemically isomeric form or a mixture of stereochemically isomeric forms thereof, or an acid addition salt thereof, wherein R¹, R², R³, R⁴ and R⁵ are as defined in formula II; with an azide compound, with the proviso that the azide does not include sodium azide, in the presence of an alcohol and a base, optionally in the presence of a first solvent, to produce a substituted cyclopropanecarbamate compound of formula IX: 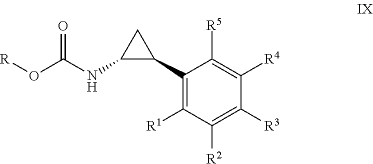
or a stereochemically isomeric form or a mixture of stereochemically isomeric forms thereof, wherein ‘R’ is an alkyl, cycloalkyl, aryl or aralkyl group; and wherein R¹, R², R³, R⁴ and R⁵ are as defined in formula II; and
b) subjecting the cyclopropanecarbamate compound of formula IX to acidic hydrolysis with an acid in a second solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.

11. The process of claim 10, wherein the alcohol used in step-(a) is selected from the group consisting of methanol, ethanol, isopropyl alcohol, isobutanol, tert-butanol, n-pentanol, cyclohexanol, 1 or d-menthol, benzyl alcohol, and mixtures thereof; wherein the first solvent used in step-(a) is selected from the group consisting of an ester, a nitrile, a hydrocarbon, a cyclic ether, an aliphatic ether, and mixtures thereof; wherein the azide used in step-(a) is selected from the group consisting of diethylphosphoryl azide, diisopropylphosphoryl azide, di-tert-butylphosphoryl azide, dibutylphosphoryl azide, dibenzylphosphoryl azide, di-1 or d-menthylphosphoryl azide and diphenylphosphoryl azide; wherein the acid used in step-(b) is selected from the group consisting of methanesulfonic acid, trifluoromethanesulfonic acid, trifluoroacetic acid, hydrochloric acid, sulfuric acid, hydrobromic acid, and mixtures thereof; and wherein the second solvent used in step-(b) is selected from the group consisting of water, an alcohol, an ester, a cyclic ether, an aliphatic ether, a hydrocarbon, and mixtures thereof.

12. The process of claim 11, wherein the first solvent is selected from the group consisting of toluene, tetrahydrofuran, 2-methyl tetrahydrofuran, and mixtures thereof; and wherein the second solvent used in step-(b) is selected from the group consisting of water, tetrahydrofuran, 2-methyl tetrahydrofuran, dioxane, and mixtures thereof.

13. A process for preparing substituted phenylcyclopropylamine derivatives of formula II: 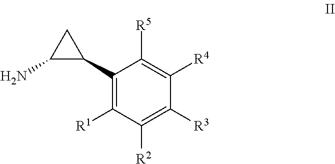
or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring of formula II is substituted with one or more halogen atoms, wherein the halogen atom is F, Cl, Br or I; comprising:
a) reacting the substituted cyclopropanecarboxylic acid compound of formula III: 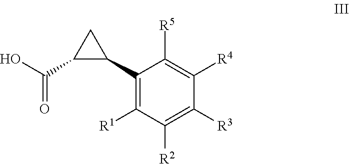
or a stereochemically isomeric form or a mixture of stereo chemically isomeric forms thereof, or an amine salt thereof, wherein R¹, R², R³, R⁴ and R⁵ are as defined in formula II; with an activating agent in the presence of a base, optionally in the presence of a racemisation suppressant, in a first solvent to produce an intermediate compound, followed by amidation with hydroxylamine or an acid addition salt thereof to produce a cyclopropanecarboxamide compound of formula X: 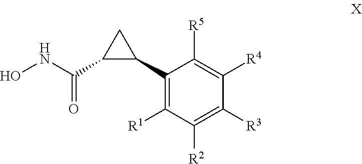
or a stereochemically isomeric form or a mixture of stereo chemically isomeric forms thereof, wherein R¹, R², R³, R⁴ and R⁵ are as defined above;
b) reacting the cyclopropanecarboxamide compound of formula X with an activating agent, followed by treatment with an alcohol, optionally in the presence of a second solvent, to produce a substituted cyclopropanecarbamate compound of formula IX: 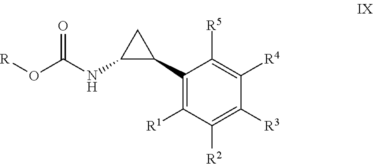
or a stereochemically isomeric form or a mixture of stereochemically isomeric forms thereof, wherein ‘R’ is an alkyl, cycloalkyl, aryl or aralkyl group; and wherein R¹, R², R³, R⁴ and R⁵ are as defined in formula II; and
c) subjecting the cyclopropanecarbamate compound of formula IX to acidic hydrolysis with an acid in a third solvent to produce the substituted phenylcyclopropylamine derivatives of formula II or a stereochemically isomeric form or a mixture of stereochemically isomeric forms thereof, and optionally converting the compound of formula II obtained into an acid addition salt thereof.

14. The process of claim 13, wherein the first solvent used in step-(a) is selected from the group consisting of water, a ketone, an ester, a hydrocarbon, a chlorinated hydrocarbon, a cyclic ether, an aliphatic ether, a nitrile, a polar aprotic solvent, and mixtures thereof; wherein the racemisation suppressant used in step-(a) is selected from the group consisting of 1-hydroxybenzotriazole, 1-hydroxy-7-azabenzotriazole, ethyl-1-hydroxy-1H-1,2,3-triazole carboxylate, N-hydroxytetrazole, 1-hydroxy-substitutedtetrazoles, 1-hydroxy-substitutedbenzo-triazines, arylphosphonium salts, and mixtures thereof; wherein the alcohol used in step-(b) is selected from the group consisting of methanol, ethanol, isopropyl alcohol, isobutanol, tert-butanol, n-pentanol, cyclohexanol, 1 or d-menthol, benzyl alcohol, and mixtures thereof; and wherein the second solvent used in step-(b) is selected from the group consisting of an ester, a nitrile, a hydrocarbon, a cyclic ether, an aliphatic ether, and mixtures thereof.

15. The process of claim 14, wherein the first solvent used in step-(a) is selected from the group consisting of acetone, dioxan, ethyl acetate, mixtures of ortho-xylene, meta-xylene, para-xylene, toluene, acetonitrile, tetrahydrofuran, dichloromethane, chloroform, methylethylketone, and mixtures thereof; wherein the racemisation suppressant used in step-(a) is 1-hydroxybenzotriazole; and wherein the second solvent used in step-(b) is selected from the group consisting of toluene, tetrahydrofuran, 2-methyl tetrahydrofuran, and mixtures thereof.

16. The process of claim 13, wherein the activating agent used in step-(a) is selected from the group consisting of 1,1-carbonyldiimidazole, 1,1′-carbonyl-di-(1,2,4-triazole), phosgene derivatives, alkyl chloro formates, arylchloroformates, 2-halo-4,6-dialkoxy-1,3,5-triazines, thionyl chloride, trialkyl phosphites, triarylphosphites, N,N-dialkylcarbodiimides, N,N-diarylcarbodiimides, diphenylphosphorylazide, 1-chloro-N,N,2-trimethyl-1-propenyl amine, chloro-N,N,N′,N′-bis(tetra-ethylene)formamidinium tetrafluoro borate, boric acid derivatives, fluoro-N,N,N′,N′-bis(tetramethylene)formamidiniumhexafluorophosphates, oxalic acid diimidazole, 2-halo-1,3-dimethylimidazolidinium chloride, 2-halo-1,3-dimethylimidazo lidinium hexafluorophosphate, benzotriazole-phosphonium salt complexes, pyrrolidinephosphoniumsalts, 3-(diethoxyphosphoryloxy)-1,2,3-benzotriazin-4(3H)-one, N/O-substituted benzotriazole salts/derivatives, O-(2-oxo-1(2H)pyridyl)-N,N,N′,N′-tetramethyluronium tetrafluoroborate, O-[(ethoxycarbonyl)cyanomethylenamino]-N,N,N′,N′-tetramethyluronium hexafluorophosphate (HOTU), 0-[(ethoxycarbonyl)cyanomethylenamino]-N,N,N′,N′-tetramethyluronium tetrafluoroborate (TOTU) and other uronium complexes, polyphosphonic anhydride, thiouronium reagents, and mixtures thereof.

17. A one pot process for preparing substituted phenylcyclopropylamine derivatives of formula II: 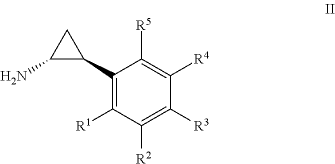
or a stereochemically isomeric form or a mixture of stereochemically isomeric forms thereof, or an acid addition salt thereof; wherein R¹, R², R³, R⁴ and R⁵ are, each independently, selected from hydrogen and a halogen atom, with the proviso that the benzene ring of formula II is substituted with one or more halogen atoms, wherein the halogen atom is F, Cl, Br or I; comprising:
a) reacting the substituted cyclopropanecarboxylic acid compound of formula III: 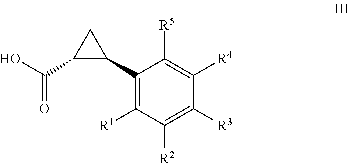
or a stereochemically isomeric form or a mixture of stereochemically isomeric forms thereof, or an amine salt thereof, wherein R¹, R², R³, R⁴ and R⁵ are as defined in formula II; with an acid activating agent in the presence of a base in a solvent to produce an intermediate compound, followed by amidation with hydroxylamine or an acid addition salt thereof to produce a cyclopropanecarboxamide compound of formula X: 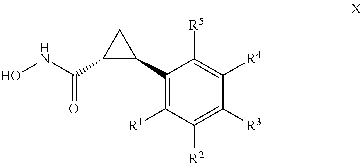
or a stereochemically isomeric form or a mixture of stereo chemically isomeric forms thereof, wherein R¹, R², R³, R⁴ and R⁵ are as defined above;
b) reacting the cyclopropanecarboxamide compound of formula X, in-situ, with a carbonyl source to produce a cyclopropanedioxazol compound of formula XI: 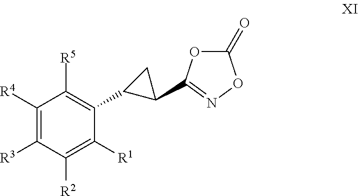
or a stereochemically isomeric form or a mixture of stereo chemically isomeric forms thereof, wherein R¹, R², R³, R⁴ and R⁵ are as defined in formula II;
c) subjecting the cyclopropanedioxazol compound of formula XI, in-situ, to thermal rearrangement at boiling temperature of the reaction solvent to produce a cyclopropaneisocyanate compound of formula XII: 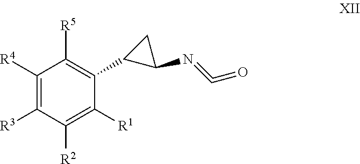
or a stereochemically isomeric form or a mixture of stereo chemically isomeric forms thereof, wherein R¹, R², R³, R⁴ and R⁵ are as defined in formula II;
d) reacting the cyclopropaneisocyanate compound of formula XII, in-situ, with an alcohol at the boiling temperature to produce a cyclopropanecarbamate compound of formula IX: 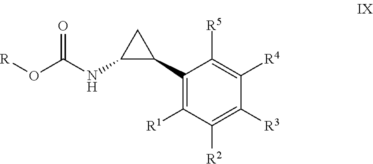
or a stereochemically isomeric form or a mixture of stereochemically isomeric forms thereof, wherein ‘R’ is an alkyl, cycloalkyl, aryl or aralkyl group; and wherein R¹, R², R³, R⁴ and R⁵ are as defined in formula II; and
e) subjecting the cyclopropanecarbamate compound of formula IX to acidic hydrolysis with an acid to produce the substituted phenylcyclopropylamine derivatives of formula II or a stereochemically isomeric form or a mixture of stereo chemically isomeric forms thereof, and optionally converting the compound of formula II obtained into an acid addition salt thereof.

18. The process of claim 17, wherein the solvent used for the one pot process is selected from the group consisting of water, a ketone, an ester, a hydrocarbon, a chlorinated hydrocarbon, a cyclic ether, an aliphatic ether, a nitrile, a polar aprotic solvent, and mixtures thereof.

19. The process of claim 18, wherein the solvent is selected from the group consisting of acetone, dioxan, ethyl acetate, mixtures of ortho-xylene, meta-xylene, para-xylene, toluene, acetonitrile, tetrahydrofuran, dichloromethane, chloroform, methylethylketone, and mixtures thereof.

20. Solid state form of an acid addition salt of trans-(1R,2S)-2-(3,4-difluorophenyl)-cyclopropylamine, wherein the acid addition salt is a tartrate salt, a di-p-toluoyl-tartrate salt, an (S)-ketopinate salt, a (D)-malate salt, a (D)-camphorsulfonate salt, a (R)-(−)-α-methoxyphenyl acetate salt, a fumarate salt, a phosphate salt or a sulfate salt.

21. The solid state form of claim 20, having the following characteristics, wherein:
1) the solid state form of trans-(1R,2S)-2-(3,4-difluorophenyl)-cyclopropylamine tartrate salt is characterized by one or more of the following properties:
i) a powder X-ray diffraction pattern substantially in accordance with FIG. 1;
ii) a powder X-ray diffraction pattern having peaks at about 5.14, 6.81, 10.32, 11.96, 12.63, 14.45, 15.34, 15.54, 15.90, 16.24, 17.50, 19.67, 20.37, 20.73 and 22.46±0.2 degrees 2-theta; and
iii) a DSC thermogram substantially in accordance with FIG. 2;
2) the solid state form of trans-(1R,2S)-2-(3,4-difluorophenyl)-cyclopropylamine di-p-toluoyl-tartrate salt is characterized by one or more of the following properties:
i) a powder X-ray diffraction pattern substantially in accordance with FIG. 3; and
ii) a powder X-ray diffraction pattern having peaks at about 6.79, 12.18, 12.57, 13.60, 14.37, 15.28, 18.21, 18.82, 19.26 and 23.40±0.2 degrees 2-theta;
3) the solid state form of trans-(1R,2S)-2-(3,4-difluorophenyl)-cyclopropylamine (S)-ketopinate salt is characterized by one or more of the following properties:
i) a powder X-ray diffraction pattern substantially in accordance with FIG. 4;
ii) a powder X-ray diffraction pattern having peaks at about 6.72, 9.49, 12.88, 13.51, 13.73, 14.37, 17.40, 17.84, 18.25, 19.14, 19.28, 19.55, 25.59, 26.23 and 27.54±0.2 degrees 2-theta; and
iii) a DSC thermogram substantially in accordance with FIG. 5;
4) the solid state form of trans-(1R,2S)-2-(3,4-difluorophenyl)-cyclopropylamine (D)-malate salt is characterized by one or more of the following properties:
i) a powder X-ray diffraction pattern substantially in accordance with FIG. 6;
ii) a powder X-ray diffraction pattern having peaks at about 5.34, 10.73, 12.79, 15.11, 16.15, 17.86, 18.78, 20.07, 21.61, 22.16, 22.30, 24.08, 27.12 and 27.46±0.2 degrees 2-theta; and
iii) a DSC thermogram substantially in accordance with FIG. 7;
5) the solid state form of trans-(1R,2S)-2-(3,4-difluorophenyl)-cyclopropylamine (D)-camphorsulfonate salt is characterized by one or more of the following properties:
i) a powder X-ray diffraction pattern substantially in accordance with FIG. 8;
ii) a powder X-ray diffraction pattern having peaks at about 6.73, 8.57, 13.89, 15.34, 16.66, 19.06, 19.62, 20.94, 24.66 and 26.70±0.2 degrees 2-theta; and
iii) a DSC thermogram substantially in accordance with FIG. 9;
6) the solid state form of trans-(1R,2S)-2-(3,4-difluorophenyl)-cyclopropylamine (R)-(−)-α-methoxyphenylacetate salt is characterized by one or more of the following properties:
i) a powder X-ray diffraction pattern substantially in accordance with FIG. 10; and
ii) a powder X-ray diffraction pattern having peaks at about 4.85, 6.63, 7.87, 9.59, 11.57, 12.43, 12.66, 15.84, 16.36, 17.53, 17.97, 18.25, 18.77, 20.11, 20.73, 21.22, 22.42, 23.09, 23.42, 25.47 and 26.94±0.2 degrees 2-theta;
7) the solid state form of trans-(1R,2S)-2-(3,4-difluorophenyl)-cyclopropylamine fumarate salt is characterized by one or more of the following properties:
i) a powder X-ray diffraction pattern substantially in accordance with FIG. 11;
ii) a powder X-ray diffraction pattern having peaks at about 4.68, 9.38, 14.09, 16.61, 18.39, 18.83, 19.82, 21.33, 22.77, 23.48, 24.30, 25.96, 26.49, 27.80 and 31.65±0.2 degrees 2-theta; and
iii) a DSC thermogram substantially in accordance with FIG. 12;
8) the solid state form of trans-(1R,2S)-2-(3,4-difluorophenyl)-cyclopropylamine phosphate salt is characterized by one or more of the following properties:
i) a powder X-ray diffraction pattern substantially in accordance with FIG. 13;
ii) a powder X-ray diffraction pattern having peaks at about 5.19, 10.39, 15.61, 21.08 and 26.17±0.2 degrees 2-theta; and
iii) a DSC thermogram substantially in accordance with FIG. 14;
9) the solid state form of trans-(1R,2S)-2-(3,4-difluorophenyl)-cyclopropylamine sulfate salt is characterized by one or more of the following properties:
i) a powder X-ray diffraction pattern substantially in accordance with FIG. 15;
ii) a powder X-ray diffraction pattern having peaks at about 4.87, 9.78, 14.72, 17.85, 18.14, 18.61, 19.31, 19.73, 21.66, 22.61, 23.93, 27.86 and 34.85±0.2 degrees 2-theta; and
iii) a DSC thermogram substantially in accordance with FIG. 16.

22. A process for the preparation of the solid state form of trans-(1R,2S)-2-(3,4-difluorophenyl)-cyclopropylamine acid addition salt of claim 20, comprising:
a) providing a first solution or suspension of trans-(1R,2S)-2-(3,4-difluorophenyl)-cyclopropylamine free base in an alcoholic solvent;
b) combining the first solution or suspension with an acid to produce a second solution or suspension containing trans-(1R,2S)-2-(3,4-difluorophenyl)-cyclopropylamine acid addition salt, wherein the acid is selected from the group consisting of tartaric acid, di-p-toluoyl-tartric acid, (S)-ketopinic acid, (D)-malic acid, (D)-camphorsulfonic acid, (R)-(−)-α-methoxyphenyl acetic acid, fumaric acid, phosphoric acid and sulfuric acid; and
c) optionally, substantially removing the solvent from the second solution or suspension to obtain a residue, followed by dissolving or suspending the residue in a second solvent to produce a third solution or suspension;
d) isolating and/or recovering the solid state form of trans-(1R,2S)-2-(3,4-difluorophenyl)-cyclopropylamine acid addition salt either from the second solution or suspension obtained in step-(b) or from the third solution or suspension obtained in step-(c).

23. The process of claim 22, wherein the alcohol solvent used in step-(a) is selected from the group consisting of methanol, ethanol, n-propanol, isopropyl alcohol, isobutanol, n-butanol, tert-butanol, amyl alcohol, isoamyl alcohol, and mixtures thereof; and wherein the second solvent used in step-(c) is selected from the group consisting of water, an alcohol, a ketone, a chlorinated hydrocarbon, a hydrocarbon, an ester, a nitrile, an ether, a polar aprotic solvent, and mixtures thereof.

24. The process of claim 23, wherein the alcohol solvent used in step-(a) is selected from the group consisting of methanol, ethanol, isopropyl alcohol, and mixtures thereof.
